(19) United States
(12) Patent Application Publication
(10) Pub. No.: US 2022/0316004 A1
(43) Pub. Date: Oct. 6, 2022

(54) DETECTION OF LABELED ANALYTES IN BIOLOGICAL SAMPLES

(71) Applicant: Akoya Biosciences, Inc., Menlo Park, CA (US)

(72) Inventors: Peter J. Miller, Cambridge, MA (US); Julia Kennedy-Darling, Redwood City, CA (US)

(21) Appl. No.: 17/714,628

(22) Filed: Apr. 6, 2022

Related U.S. Application Data
(60) Provisional application No. 63/171,297, filed on Apr. 6, 2021.

(51) Int. Cl. C12Q 1/6874; C12Q 1/6825

(52) U.S. Cl. CPC C12Q 1/6874; C12Q 1/6825

(57) ABSTRACT

Methods for determining locations of analytes include: (a) exposing a biological sample to a plurality of different types of probes, where each different type of probe includes a nucleic acid capture moiety and a detection moiety that includes at least one reporter moiety, where the at least one reporter moiety features multiple label regions, each of the label regions including an oligonucleotide having a sequence; (b) exposing the biological sample to a plurality of optical labels; (c) measuring optical signals generated by optical labels; (d) repeating steps (b) and (c) with different pluralities of optical labels; (e) identifying one or more of the reporter moieties in the sample based on the measured optical signals; and (f) determining a location of one or more of the RNA analytes in the sample based on the identified reporter moieties.

### CROSS-REFERENCE TO RELATED APPLICATIONS

This application claims priority to U.S. Provisional Patent Application No. 63/171,297, filed on Apr. 6, 2021, the entire contents of which are incorporated herein by reference.

### TECHNICAL FIELD

This disclosure relates to labeling analytes in biological samples with detectable reporter moieties, detecting the labeled analytes, and compensating for a variety of detection errors.

### BACKGROUND

Fluorescence imaging can be used to detect and quantify a wide variety of different analytes in biological samples. By labeling each different type of analyte with a different fluorescent dye, multiplexed detection of multiple analytes can be performed. In some assays, the number of different analytes that can be detected is limited by the number of distinct fluorescence measurement signals that can be resolved.

### SUMMARY

This disclosure features methods, systems, and kits for labeling analytes in biological samples, measuring signals corresponding to the labeled analytes, detecting and quantifying the labeled analytes, and correcting for errors in analyte labeling and measured signals. Analytes are each labeled with multiple reporter moieties, and reporter moieties used to label each different type of analyte are selected such that by measuring signals corresponding to the reporter moieties, each different type of analyte can be unambiguously detected in the sample. Further, measurement errors such as signals that are not detected can be identified and corrected such that the corresponding labeled analytes can still be reliably quantified. The methods, systems, and kits can be used with a wide variety of different types of reporter moieties and sample analytes. Further, by selecting both the number of distinct reporter moieties that are applied to a sample and the number of reporter moieties that are used to label individual analytes, the number of different analytes that can be distinctly and reliably detected can be adjusted. With even a relatively modest number of distinct reporter moieties, a relatively large number of different analytes can be detected.

In an aspect, the disclosure features methods that include: (a) exposing a biological sample to a plurality of different types of probes, where each different type of probe includes a nucleic acid capture moiety that binds to a different type of RNA analyte in the sample, and a detection moiety featuring at least one reporter moiety, where the detection moiety is linked to the capture moiety, and where the at least one reporter moiety includes multiple label regions, each of the label regions featuring an oligonucleotide having a sequence; (b) exposing the biological sample to a plurality of optical labels, where each of the optical labels includes an oligonucleotide having a sequence, and a species that generates an optical signal; (c) measuring optical signals generated by optical labels of the plurality of optical labels that hybridize to complementary label regions of the reporter moieties; (d) repeating steps (b) and (c) with different pluralities of optical labels; (e) identifying one or more of the reporter moieties in the sample based on the measured optical signals; and (f) determining a location of one or more of the RNA analytes in the sample based on the identified reporter moieties, where the at least one reporter moiety of each type of detection moiety differs from the at least one reporter moiety of each other type of detection moiety among the different types of probes by at least two label regions.

Embodiments of the methods can include any one or more of the following features.

The methods can include, for at least one of the optical labels, removing the at least one of the optical labels from the sample after measuring an optical signal generated by the at least one of the optical labels and before repeating steps (b) and (c) with different pluralities of optical labels. Removing the at least one of the optical labels can include dehybridizing the at least one of the optical labels from one or more label regions of the reporter moieties.

The at least one reporter moiety of at least one type of detection moiety can include label regions that do not repeat within the at least one reporter moiety. The at least one reporter moiety of each type of detection moiety can include label regions that do not repeat within the at least one reporter moiety.

The at least one reporter moiety of at least one type of detection moiety can include at least 3 label regions that do not repeat within the at least one reporter moiety. The at least one reporter moiety of each type of detection moiety can include at least 3 label regions that do not repeat within the at least one reporter moiety.

Each label region can include at least 15 nucleotides (e.g., at least 30 nucleotides). Each label region of each of the reporter moieties can include a same number of nucleotides. Each of the reporter moieties can include a same number of label regions.

The species that generates the optical signal can be a fluorescent moiety. The species that generates the optical signal can include at least one fluorescent nucleotide. Measuring optical signals generated by the optical labels can include obtaining at least one image of the optical labels in the sample, and identifying optical signals corresponding to the optical labels in the at least one image.

Each plurality of optical labels in step (b) can include a same number of different types of optical labels. Each plurality of optical labels in step (b) can include 3 or more (e.g., 5 or more) different types of optical labels. The methods can include repeating step (b) until the sample has been exposed to a set of optical labels, where each label region of the at least one reporter moiety has a complementary optical label in the set of optical labels.

The methods can include exposing the sample to at least one of the plurality of optical labels more than once.

Each time step (b) is performed, each member of the plurality of optical labels in step (b) can include a species that generates a different optical signal. The different optical signals can have different spectral distributions.

Among the plurality of optical labels, at least two of the optical labels can include a common species that generates the optical signal, and the at least two of the optical labels can be exposed to the sample during different repetitions of step (b). Among the plurality of optical labels, first and second optical labels can each include a first species that generates the optical signals of the first and second optical labels, third and fourth optical labels can each include a second species that generates the optical signals of the third and fourth optical labels, and the first and second species can be different. The optical signals generated by the first and second species can be different. The methods can include exposing the sample to the first and second optical labels during different repetitions of step (b), and exposing the sample to the third and fourth optical labels during different repetitions of step (b).

Embodiments of the methods can also include any of the other features disclosed herein, and can include any combination of such features, whether or not such features are described in a common embodiment or in different embodiments, except as expressly stated otherwise.

In another aspect, the disclosure features methods that include: exposing a biological sample to a first probe that includes a first nucleic acid capture moiety that binds to a first RNA in the sample, and a first detection moiety that includes at least one first reporter moiety, where the at least one first reporter moiety features multiple first label regions each including an oligonucleotide having a sequence, where the oligonucleotide sequences of each of the multiple first label regions in each first reporter moiety are different; exposing the biological sample to a second probe that includes a second nucleic acid capture moiety that binds to a second RNA in the sample, and a second detection moiety that includes at least one second reporter moiety, where the at least one second reporter moiety features multiple second label regions each including an oligonucleotide having a sequence, where the oligonucleotide sequences of each of the multiple second label regions in each second reporter moiety are different; exposing the biological sample to multiple pluralities of optical labels, where each plurality of optical labels includes at least one optical label that hybridizes to at least one of the multiple first and second label regions, until optical labels from the multiple pluralities of optical labels have hybridized to each of the multiple first and second label regions; after exposing the biological sample to each plurality of optical labels, measuring spatially resolved optical signals generated by the at least one optical label of each plurality of optical labels that hybridizes to at least one of the multiple first and second label regions; and determining one or more locations of the first and second RNAs in the sample, where at least two of the first label regions are different from each of the second label regions.

Embodiments of the methods can include any one or more of the following features.

The methods can include, for at least one of the pluralities of optical labels, removing the at least one optical label that hybridizes to at least one of the multiple first and second label regions from the sample after measuring spatially resolved optical signals and before exposing the biological sample to another one of the multiple pluralities of optical labels. Removing the at least one optical label can include dehybridizing the at least one optical label from the at least one of the multiple first and second label regions.

Each reporter moiety of the at least one first reporter moiety can include first label regions that do not repeat within the reporter moiety. Each reporter moiety of the at least one second reporter moiety can include second label regions that do not repeat within the reporter moiety. Each reporter moiety of the at least one first reporter moiety can include at least 3 first label regions that do not repeat within the reporter moiety. Each reporter moiety of the at least one second reporter moiety can include at least 3 second label regions that do not repeat within the reporter moiety.

Each label region can include at least 15 nucleotides (e.g., at least 30 nucleotides). Each label region of the at least one first reporter moiety can include a same number of nucleotides. Each of the at least one first reporter moieties can include a same number of label regions. Each of the at least one second reporter moieties can include the same number of label regions.

Each of the optical labels can include a species that generates an optical signal. The species that generates the optical signal can be a fluorescent moiety. The species that generates the optical signal can include at least one fluorescent nucleotide. Measuring spatially resolved optical signals generated by the at least one optical label can include obtaining at least one image of the at least one optical label in the sample, and identifying optical signals corresponding to the optical labels in the at least one image.

Each plurality of optical labels can include a same number of different types of optical labels. Each plurality of optical labels can include 3 or more different types of optical labels (e.g., 5 or more different types of optical labels). At least one type of optical label can be present in more than one of the multiple pluralities of optical labels. The optical labels of each plurality of optical labels can include respective species that generate different optical signals from one another. The different optical signals can have different spectral distributions.

Among the multiple pluralities of optical labels, at least two types of the optical labels can include a common species that generates a common optical signal, and the at least two types of optical labels can be present in different pluralities of optical labels. The biological sample can be exposed to the different pluralities at different times.

Embodiments of the methods can also include any of the other features disclosed herein, and can include any combination of such features, whether or not such features are described in a common embodiment or in different embodiments, except as expressly stated otherwise.

In a further aspect, the disclosure features compositions that include multiple pluralities of probes, where each plurality of probes targets a different type of analyte and includes a capture moiety that binds to the type of analyte targeted by the plurality of probes and a detection moiety linked to the capture moiety and comprising at least one reporter moiety, where the at least one reporter moiety includes multiple label regions each featuring an oligonucleotide having a sequence, where for each plurality of probes, the label regions of the at least one reporter moiety differ from the label regions of the at least one reporter moiety of the other pluralities of probes by at least two label regions.

Embodiments of the compositions can include any one or more of the following features.

For at least one of the pluralities of probes, the label regions of the at least one reporter moiety can be each different from one another. For each plurality of probes, the label regions of the at least one reporter moiety can be each different from one another. The multiple pluralities of probes can include 50 or more pluralities of probes (e.g., 500 or more pluralities of probes).

Each plurality of probes can target a different RNA. The capture moiety of each plurality of probes can include an oligonucleotide having a sequence that is at least partially complementary to a RNA analyte. For at least one of the pluralities of probes, the multiple label regions of the at least one reporter moiety can include at least 3 label regions (e.g., at least 4 label regions). For each of the pluralities of probes, the multiple label regions of the at least one reporter moiety can include at least 3 label regions.

For at least one of the pluralities of probes, the oligonucleotide sequences of the multiple label regions of the at least one reporter moiety can have a common length. For each of the pluralities of probes, the oligonucleotide sequences of the multiple label regions of the at least one reporter moiety can have a common length. For at least one of the pluralities of probes, the oligonucleotide sequences of the multiple label regions of the at least one reporter moiety can vary in length.

For at least one of the pluralities of probes, the oligonucleotide sequences of the multiple label regions of the at least one reporter moiety can have a common length that is different from a common length of the oligonucleotide sequences of the multiple label regions of the at least one reporter moiety of another one of the pluralities of probes.

For each plurality of probes, each of the multiple label regions of the at least one reporter moiety can include at least 15 nucleotides (e.g., at least 30 nucleotides). For each plurality of probes, the label regions of the at least one reporter moiety can differ from the label regions of the at least one reporter moiety of the other pluralities of probes by at least three label regions.

Each plurality of probes can target a different peptide. The capture moiety of each plurality of probes can include an antibody that selectively binds a protein analyte.

At least one of the pluralities of probes can include probes that selectively bind to a RNA, and at least one of the pluralities of probes can include probes that selectively bind to a protein.

Embodiments of the compositions can also include any of the other features disclosed herein, and can include any combination of such features, whether or not such features are described in a common embodiment or in different embodiments, except as expressly stated otherwise.

In another aspect, the disclosure features kits that include a packaging material and, within the packaging material, any of the compositions disclosed herein.

Embodiments of the kits can include any one or more of the following features.

The composition can be a first composition, and the kits can include a second composition within the packaging material and that includes a plurality of optical labels, where each optical label features an oligonucleotide sequence and a species that generates an optical signal. The oligonucleotide sequences of each of the optical labels can be different. At least some of the species that generate the optical signal can be common to multiple optical labels.

The oligonucleotide sequence of each optical label can include at least 15 nucleotides (e.g., at least 30 nucleotides). The species that generates the optical signal can include a fluorescent moiety. The species that generates the optical signal can include a dye. The species that generates the optical signal can include at least one fluorescent nucleotide.

The oligonucleotide sequence of each optical label can have a common length. At least some of the oligonucleotide sequences of the optical labels can have different lengths. The oligonucleotide sequences of the optical labels can be DNA sequences.

The optical labels can be packaged in multiple groups within the packaging material.

Embodiments of the kits can also include any of the other features disclosed herein, and can include any combination of such features, whether or not such features are described in a common embodiment or in different embodiments, except as expressly stated otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features and advantages will be apparent from the description, drawings, and claims.

### DESCRIPTION OF DRAWINGS

FIG. 1 is a schematic diagram of an example of a labeled analyte in a biological sample.

FIG. 2A is a schematic diagram of one example of a reporter moiety.

FIG. 2B is a schematic diagram of another example of a reporter moiety.

FIG. 2C is a schematic diagram of a further example of a reporter moiety.

FIG. 3 is a flow chart showing a series of example steps for detecting analytes in a sample via multiple cycles of hybridization of optical labels.

FIG. 4A is a schematic diagram showing an example set of detection cycles for identifying an analyte in a sample.

FIG. 4B is a schematic diagram showing two different types analytes in a sample.

FIG. 4C is a schematic image of the sample of FIG. 4B showing locations of the two different types of analytes.

FIG. 4D is a schematic diagram showing optical signals measured in each of 6 detection cycles performed on a sample containing both of the different analytes of FIG. 4B.

FIG. 5 is a schematic diagram of an example of a probe,

FIG. 6 is a schematic diagram of another example of a probe.

FIG. 7 is a schematic diagram of a system for analyzing a biological sample.

FIG. 8 is a schematic diagram of a controller.

Like reference symbols in the various drawings indicate like elements.

### DETAILED DESCRIPTION

### Introduction

Multiplexed fluorescence detection is a widely known technique for detecting proteins, nucleic acids, and other analytes in biological samples. Conventional fluorescence imaging methods involve applying different fluorescent dyes to different analytes in a sample, and then exposing the dyes to incident light to cause each dye to emit fluorescence. By spectrally resolving fluorescence emission signals from the different dyes, analytes in the sample can be identified and quantified. When the fluorescence emission signals are also spatially resolved as in a multispectral fluorescence image, information about the presence and abundance of multiple different analytes at multiple locations in a sample can be obtained. This information is valuable for a wide variety of research and clinical diagnostic purposes, including identifying markers of disease, elucidating intracellular communications pathways, assessing gene expression and cellular regulation activity, and monitoring the effectiveness of pharmaceutical and other therapeutic trials and interventions.

In conventional fluorescence imaging methods, the number of analytes that can be detected can be limited by the number of different measurement signals that can be detected and resolved. Methods exist for separating fluorescence emission signals from dyes that overlap spectrally, even when the overlap is significant. For example, eigenvector decomposition methods such as spectral unmixing can be used to separate such signals, provided that relatively good estimates for the pure spectra of the individual fluorescent dyes (i.e., the eigenvectors) are available or measurable. Even with such techniques, however, the number of different measurement signals that can be resolved—and the corresponding number of different analytes that can be quantified—may be relatively limited due to spectral congestion of the dyes involved.

Limits on the number of different analytes may not represent significant drawbacks for certain types of assays. For example, for certain tumor-specific assays that target a relatively small number of expressed cell markers via antibody-based probes that selectively bind to the markers, the foregoing limits may not represent a significant shortcoming. However, assays that target a more diverse set of cell markers, proteomics assays designed to obtain more general information about expressed proteins in cells, and nucleic acid assays that target cellular RNA and DNA may be designed to quantify tens, hundreds, or thousands of distinct peptides and/or transcripts. For these expansive assays, the above limitations may represent significant roadblocks.

One approach to increasing the number of distinct analytes that can be detected and quantified involves using reporter moieties with spectrally narrow and distinct emission characteristics. For example, quantum dot-based reporter moieties can be synthesized and incorporated into probes that bind specifically to different types of analytes. By controlling the composition and size distribution of the quantum dots, populations of different reporter moieties with (spectrally) closely-spaced but resolvable fluorescence emission signals can be obtained. Incorporated into different types of probes, the resolvable reporter moieties can be used to identify a larger number of different analytes than would typically be possible with conventional fluorescent dye-based reporter moieties.

However, as the number of different analytes increases, the complexity associated with synthesizing populations of distinct reporting moieties increases, as does the time required to synthesize and characterize the reporter moieties prior to use. For an assay involving several hundred different analytes (i.e., peptide or nucleic acid analytes such as RNA transcripts), the synthetic burdens discussed above may be too significant to make such approaches practical and cost-effective.

Instead of assigning a different “color” to each type of analyte via a distinct reporter moiety that is specifically bound only to analytes of that type, the methods described herein involve labeling individual analytes with combinations of different reporter moieties. In particular, each different type of analyte is labeled with a distinct combination of reporter moieties, such that detection of signals generated by the reporter moieties, taken in combination, uniquely identify the different types of analytes. By labeling analytes with combinations of reporter moieties, comparatively fewer individually distinct reporter moieties are needed to distinguish among a set of different analytes. As a result, the spectral congestion and difficulties associated with resolving spectrally closely-spaced emission signals can be substantially reduced. As a consequence, a much larger number of different analytes can be reliably detected and quantified. Further, existing sets of probes that are designed to target particular groups of analytes can be more readily augmented to include probes for additional analytes without resorting to complex synthetic methods to produced tightly controlled distributions of spectrally distinct reporter moieties.

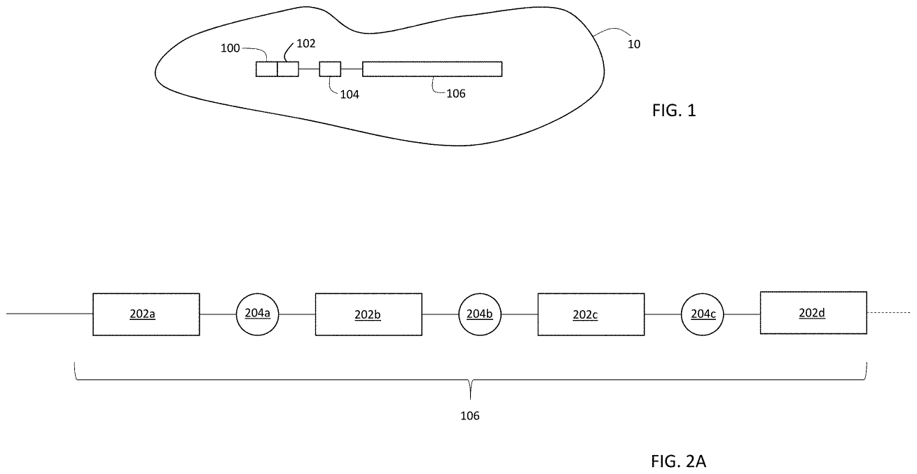

FIG. 1 is a schematic diagram showing a labeled analyte 100 in a biological sample 10. Analyte 100 is bound to a capture moiety 102 that is conjugated to a reporter moiety 106 through a linker 104. Biological sample 10 can be any one of a variety of different types of samples. Examples of biological sample 10 include, but are not limited to, tissue sections (e.g., fresh sections, fresh-frozen sections, formalin-fixed paraffin embedded sections), tissue biopsies, cells, cell suspensions, cell dispersions, cell cultures, and various bodily fluids such as blood, urine, interstitial fluid, and lymphatic fluid.

In some embodiments, biological sample 10 can be immobilized on a surface. For example, the surface can be a surface of a slide, a plate, a well, a tube, a membrane, or a film. In some embodiments, biological sample 10 can be mounted on a slide. In certain embodiments, biological sample 10 can be fixed using a fixative, such as an aldehyde, an alcohol, an oxidizing agent, a mercurial, a picrate, HOPE fixative, or another fixative. Biological sample 10 may alternatively, or in addition, be fixed using heat fixation. Fixation can also be achieved via immersion or perfusion.

Analyte 100 can be any of a variety of different analytes. In some embodiments, analyte 100 is an RNA species. In certain embodiments, analyte 100 is a DNA species. Other examples of analyte 100 include, but are not limited to, antigens, peptides, proteins, and other amino-acid containing moieties, and oligonucleotides, including oligonucleotides containing DNA bases, RNA bases, both DNA and RNA bases, and synthetic bases, nucleic acid fragments, and lipids.

Analyte 100 can be a clinically relevant biomarker, particularly a biomarker that is expressed in tumor tissue, in the tumor microenvironment, and tissues representative of other disease states. Examples of such biomarkers include, but are not limited to: tumor markers such as Sox10, S100, pan-cytokeratin, PAX5, PAX8; immune cell identifiers such as CD3, CD4, CD8, CD20, FoxP3, CD45RA, CD45LCA, CD68, CD163, CD11c, CD33, HLADR; activation markers such as Ki67, granzyme B; and checkpoint-related markers such as TIM3, LAG3, PD1, PDL1, CTLA4, CD80, CD86, IDO-1, VISTA, CD47, CD26.

Capture moiety 102 selectively binds to analyte 100 in sample 10 to attach reporter moiety 106 to the analyte. A variety of different reversible and irreversible binding mechanisms can occur between capture moiety 102 and analyte 100. In certain embodiments, where analyte 100 is an RNA or DNA, capture moiety 102 can be a RNA or DNA that is at least partially complementary to analyte 100, and binds to analyte 100 by hybridizing to analyte 100. In some embodiments, where capture moiety 102 is an antibody or antibody fragment and analyte 100 is an antigen, binding occurs between the antigen epitope and the paratope of the antibody or antibody fragment. In certain embodiments, where capture moiety 102 is an antibody or antibody fragment and analyte 100 is a lipid, binding can occur between a recognition site on the antibody or antibody fragment and a head group of the lipid (e.g., a phospholipid head group). In some embodiments, binding between capture moiety 102 and analyte 100 can occur via the formation of one or more covalent bonds. Alternatively, or in addition, binding between capture moiety 102 and analyte 100 can occur via the formation of one or more non-covalent bonds. Various fixing agents can be used to promote the formation of covalent and or non-covalent bonds.

Linker 104 can be implemented in a variety of different ways. In some embodiments, for example, linker 104 is a non-covalent or covalent bond. That is, capture moiety 102 and reporter moiety 106 can be linked directly via a bond, with no intervening moiety between them.

In certain embodiments, linker 104 can be implemented as a primary-secondary antibody pair. To label analyte 100 with reporter moiety 106, analyte 100 is exposed to a first labeling agent that includes capture moiety 102 conjugated to a primary antibody, which functions as a part of linker 104. Once the first labeling agent selectively binds to analyte 100, a second labeling agent is introduced that includes reporter moiety 106 conjugated to a secondary antibody that functions as another part of linker 104. The secondary antibody selectively binds to the first antibody, forming a linker 104 consisting of the associated antibodies, and labeling analyte 100 with reporter moiety 106.

In some embodiments, linker 104 can be implemented as a double-stranded nucleic acid (e.g., hybridized nucleic acid strands that are at least partially complementary). To label analyte 100 with reporter moiety 106, analyte 100 is exposed to a first labeling agent that includes capture moiety 102 linked to a first nucleic acid, which functions as a part of linker 104. Once the first labeling agent selectively binds to analyte 100, a second labeling agent is introduced that includes reporter moiety 106 linked to a second nucleic acid that functions as another part of linker 104. The second nucleic acid is at least partially complementary to the first nucleic acid, and selectively hybridizes to the first nucleic acid, labeling analyte 100 with reporter moiety 106.

In the foregoing example, capture moiety 102 can be linked to the first nucleic acid through conjugation, e.g., capture moiety 102 can be covalently bonded to the first nucleic acid. Alternatively, the first nucleic acid can be a nucleic acid sequence that is contiguous with capture moiety 102. That is, a nucleic acid molecule (e.g., a single-stranded or partially double-stranded nucleic acid molecule) can include a capture region consisting of a capture nucleic acid sequence that functions as capture moiety 102, and a linking region consisting of a linking nucleic acid sequence that functions as the first nucleic acid of linker 104.

In the same manner, reporter moiety 106 can be linked to the second nucleic acid through covalent bonding. Alternatively, the second nucleic acid can be a nucleic acid sequence that is contiguous with reporter moiety 106. In other words, a nucleic acid molecule (e.g., a single-stranded or partially double-stranded nucleic acid molecule) can include a linking region consisting of a linking nucleic acid sequence that functions as the second nucleic acid of linker 104, and a reporter region that includes the label regions described herein that form reporter moiety 106.

In some embodiments, the first and second nucleic acids that function as portions of linker 104 do not directly hybridize. Instead, the first and second nucleic acids each hybridize to a portion of a bridging oligonucleotide that includes nucleic acid sequences that are at least partially complementary to each of the first and second nucleic acids. Bridging oligonucleotides can be linear such that a single capture moiety is linked to a single reporter moiety. Alternatively, bridging oligonucleotides can be branched, and can include a single nucleic acid sequence that hybridizes to capture moiety 102, and multiple nucleic acid sequences that hybridize to reporter moieties. As a result, a single capture moiety 102 can be linked to multiple reporter moieties 106, allowing for amplification of optical signals that correspond to the analyte to which capture moiety 102 selectively binds.

In certain embodiments, linker 104 can be implemented as any of a variety of aliphatic and/or aromatic linking species. Examples of such species include, but are not limited to, C₁₋₂₀ cyclic and non-cyclic alkyl species, C₂₋₂₀ cyclic and non-cyclic alkene species, C₂₋₂₀ cyclic and non-cyclic alkyne species, and C₃₋₂₄ aromatic species. Any of the foregoing species can include heteroatoms such as, but not limited to, O, S, N, and P. Any of the foregoing species can also include one or more substituents selected from the group consisting of: halide groups; nitro groups; azide groups; hydroxyl groups; primary, secondary, and tertiary amine groups; aldehyde groups; ketone groups; amide groups; ether groups; ester groups; thiocyanate groups; and isothiocyanate groups.

In general, reporter moiety 106 includes two or more different label regions. Each label region of reporter moiety 106 binds to an optical label that generates an optical signal. For example, in some embodiments, each label region includes a nucleic acid sequence (e.g., a DNA sequence or an RNA sequence).

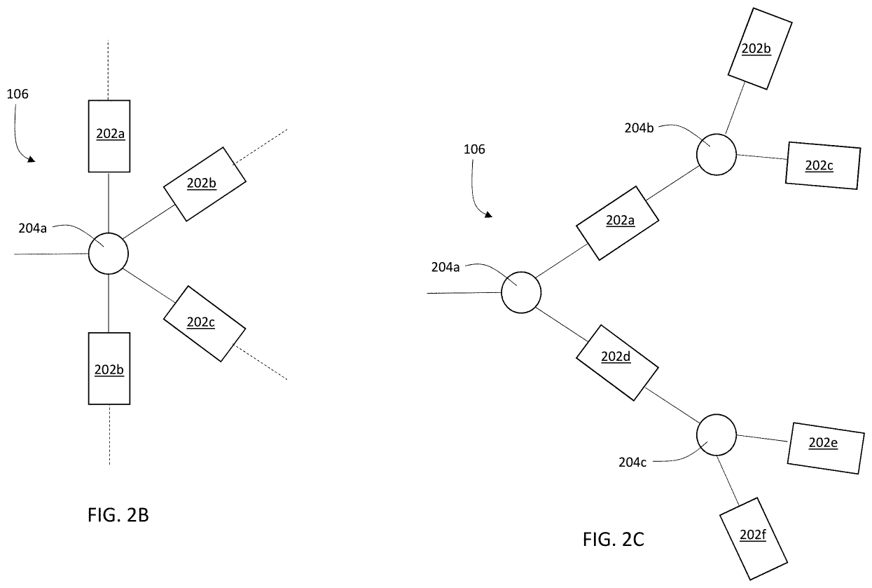

Reporter moieties 106 can generally have a variety of structures that incorporate multiple label regions. FIG. 2A shows an example of a reporter moiety 106 that includes four different label regions 202a-d, linked in a linear geometry through linkers 204a-c. In general, linkers 204a-c can individually correspond to any of the linkers 104 described above. Linkers 204a-c can each be the same, or one or more of the linkers 204a-c can be different from the others.

In particular, in some embodiments, one or more of the linkers within reporter moiety 106 is a non-covalent or covalent bond. That is, one or more of the label regions in reporter moiety 106 can be linked directly via a bond, with no intervening moiety between them.

In certain embodiments, one or more of the linkers within reporter moiety 106 is implemented as a primary-secondary antibody pair. For example, one label region can be conjugated to a primary antibody, which functions as a part of the linker. Another label region is conjugated to a secondary antibody that functions as another part of the linker. The secondary antibody selectively binds to the first antibody, forming a linker consisting of the associated antibodies, and linking the two label regions.

In some embodiments, one or more of the linkers within reporter moiety 106 can be implemented as a double-stranded nucleic acid (e.g., hybridized nucleic acid strands that are at least partially complementary). One label region can be linked to a first nucleic acid, which functions as a part of the linker, and a second label region can be linked to a second nucleic acid that functions as another part of the linker. The second nucleic acid is at least partially complementary to the first nucleic acid, and selectively hybridizes to the first nucleic acid, linking the first and second label regions.

In the foregoing example, either or both of the first and second label regions can be linked to first and second nucleic acids, respectively, through conjugation, e.g., covalent bonding. Alternatively, the first nucleic acid can be a nucleic acid sequence that is contiguous with the first label region. That is, a nucleic acid molecule (e.g., a single-stranded or partially double-stranded nucleic acid molecule) can include a label region, and a linking region consisting of a linking nucleic acid sequence that functions as the first nucleic acid of the linker.

In the same manner, the second label region can be linked to the second nucleic acid through covalent bonding. Alternatively, the second nucleic acid can be a nucleic acid sequence that is contiguous with the second label region. In other words, a nucleic acid molecule (e.g., a single-stranded or partially double-stranded nucleic acid molecule) can include a linking region consisting of a linking nucleic acid sequence that functions as the second nucleic acid of the linker, and the second label region.

In some embodiments, the first and second nucleic acids that function as portions of a linker between label regions do not directly hybridize. Instead, the first and second nucleic acids each hybridize to a portion of a bridging oligonucleotide that includes nucleic acid sequences that are at least partially complementary to each of the first and second nucleic acids.

In certain embodiments, one or more of the linkers within reporter moiety 106 can be implemented as any of a variety of aliphatic and/or aromatic linking species. Examples of such species include, but are not limited to, C₁₋₂₀ cyclic and non-cyclic alkyl species, C₂₋₂₀ cyclic and non-cyclic alkene species, C₂₋₂₀ cyclic and non-cyclic alkyne species, and C₃₋₂₄ aromatic species. Any of the foregoing species can include heteroatoms such as, but not limited to, O, S, N, and P. Any of the foregoing species can also include one or more substituents selected from the group consisting of: halide groups; nitro groups; azide groups; hydroxyl groups; primary, secondary, and tertiary amine groups; aldehyde groups; ketone groups; amide groups; ether groups; ester groups; thiocyanate groups; and isothiocyanate groups.

FIG. 2B shows another example of a reporter moiety 106 in which four different label regions 202a-d are conjugated to a common linker 204a. Linker 204a can correspond to any of the linkers 104 described above. In addition, linker 204a can correspond to a bead or particle to which label regions 202a-d are conjugated.

FIG. 2C shows a further example of a reporter moiety 106 in which six different label regions 202a-202f are conjugated to linkers 204a-c. In FIG. 2C, linkers 204b-c are each individually conjugated to three label regions, while linker 204a is conjugated to two label regions 202a, 202d, and also directly or indirectly to capture moiety 102. Linkers 204a-c can each correspond to any of the different types of linkers described above. Linkers 204a-c can each be the same, or one or more of linkers 204a-c can be different.

While the examples shown in FIGS. 2A-2C include 4 or 6 different label regions, more generally each reporter moiety 106 can include 2 or more (e.g., 3 or more, 4 or more, 5 or more, 6 or more, 7 or more, 8 or more, 9 or more, 10 or more, 12 or more, 15 or more, 20 or more, or even more) different label regions.

In some embodiments, each of the label regions in reporter moiety 106 is different (i.e., has a different nucleic acid sequence). Alternatively, in certain embodiments, one or more of the label regions in reporter moiety 106 has a sequence that is common to another one of the label regions (i.e., they repeat) in reporter moiety 106. The number of label regions that repeat in a reporter moiety can be 2 or more (e.g., 3 or more, 4 or more, 5 or more, 6 or more, 7 or more, 8 or more). Label regions that are the same in a reporter moiety 106 can be used for signal amplification and/or to compensate for detection errors arising from mis- (or no) hybridization of optical labels.

In some embodiments, each label region includes multiple nucleotide bases forming a nucleic acid sequence. The label regions of a reporter moiety 106 can each have the same number of nucleotides, or one or more of the label regions can have a different number of nucleotides. In certain embodiments, each of the label regions has the same number of nucleotides. In some embodiments, each of the label regions has a different number of nucleotides.

Each label region can independently include DNA bases (e.g., A, C, G, T), RNA bases (e.g., A, C, G, U), and any combination of DNA and/or RNA bases. Each label region can also include non-natural (e.g., synthetic) nucleotides, including DNA analogues and/or RNA analogues. Examples of such synthetic analogues include, but are not limited to, peptide nucleic acids (PNAs), morpholino and locked nucleic acids (LNAs), glycol nucleic acids, and threose nucleic acids.

The length of each label region (e.g., the number of nucleotides in a label region) can generally be selected as desired to ensure efficient and selective hybridization with an optical label. In some embodiments, each label region can include at least 5 (e.g., at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100) nucleotides.

In some embodiments, each label region can have between 5-30, between 5-25, between 5-20, between 10-20, between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides.

In certain embodiments, each label region can have no more than 5 (e.g., no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100) nucleotides.

In some embodiments, each label region can be fully single stranded. Alternatively, in certain embodiments, one or more label regions can be at least partially double stranded. A partially double stranded portion of a label region can be at the 3′ end of the label region, at the 5′ end of the label region, or between the 5′ end and 3′ ends of the label region.

In some embodiments, each reporter moiety 106 bound to an analyte 100 in sample 10 includes the same number of label regions. In certain embodiments, however, at least one reporter moiety 106 bound to an analyte in sample 10 includes a different number of label regions from the other reporter moieties 106 bound to analytes in the sample. In certain embodiments, a sample 10 can include 2 or more (e.g., 3 or more, 4 or more, 5 or more, 6 or more, or even more) groups of reporter moieties, where the reporter moieties within each group have a different number of label regions from the reporter moieties in the other groups. Each reporter moiety of each group is bound to a different analyte in the sample, as discussed above.

With one or more analytes 100 in sample 10 each bound to a different reporter moiety 106 through a capture moiety 102, the analytes in the sample can be detected and quantified through successive cycles of optical label binding and imaging in the sample. For analytes bound to reporter moieties 106 that include oligonucleotide label regions, optical labels that include an oligonucleotide conjugated to a species that generates an optical signal can be hybridized to the oligonucleotide label regions. By measuring the optical signals generated by each optical label, individual analytes in the sample can be identified and quantified, as each different type of analyte in the sample is conjugated to a different type of reporter moiety, and therefore the combination of optical signals attributable to the optical labels that hybridize to each type of reporter moiety is unique.

Where the label regions of reporter moiety 106 are oligonucleotide-based, the optical labels that are used in the methods described herein generally also include an oligonucleotide. The oligonucleotides of the optical labels each have a sequence that is at least partially, or fully, complementary to one of the label regions of a reporter moiety 106 conjugated to an analyte in the sample. Oligonucleotides of the optical labels can each have the same or a different number of bases. More generally, the oligonucleotides of the optical labels can have the same properties as the oligonucleotides of the label regions discussed above.

In general, each optical label includes at least one species that generates an optical signal, which is referred to herein as a “dye.” That is, a dye is a moiety that interacts with incident light, and from which emitted light can be measured and used to detect the presence of the dye in a sample. In general, a dye can be a fluorescent moiety, an absorptive moiety (e.g., a chromogenic moiety), or another type of moiety that emits light, or modifies incident light passing through or reflected from a sample where the dye is present so that the presence of the dye can be determined by measuring changes in transmitted or reflected light from the sample.

In certain embodiments, the optical label can include a hapten. The hapten can subsequently (or concurrently) be bound to a dye to provide a labeling moiety that can be detected by measuring emitted, transmitted, or reflected light from the sample.

A wide variety of different dyes can generally be used in optical labels. For example, the dye can be a xanthene-based dye, such as a fluorescein dye and/or a rhodamine dye. Examples of suitable fluorescein and rhodamine dyes include, but are not limited to, fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110.

The dye can also be a cyanine-based dye. Suitable examples of such dyes include, but are not limited to, the dyes Cy3, Cy5 and Cy7. The dye can also be a coumarin dye (e.g., umbelliferone), a benzimide dye (e.g., any of the Hoechst dyes such as Hoechst 33258), a phenanthridine dye (e.g., Texas Red), an ethidium dyes, an acridine dyes, a carbazole dye, a phenoxazine dye, a porphyrin dye, a polymethine dye (e.g., any of the BODIPY dyes), and a quinoline dye.

When the dye is a fluorescent species, the dye can be a moiety corresponding to any of the following non-limiting examples and/or derivatives thereof: pyrenes, coumarins, diethylaminocoumarins, FAM, fluorescein chlorotriazinyl, fluorescein, RI 10, JOE, R6G, tetramethylrhodamine, TAMRA, lissamine, napthofluorescein, Texas Red, Cy3, and Cy5.

In certain embodiments, the dye can include one or more quantum dot-based species. Quantum dot-based fluorophores are available with fluorescence emission spectra in many different spectral bands, and suitable quantum dot-based dyes can be used as labeling species in the methods described herein.

### Multiple-Cycle Detection of Analytes

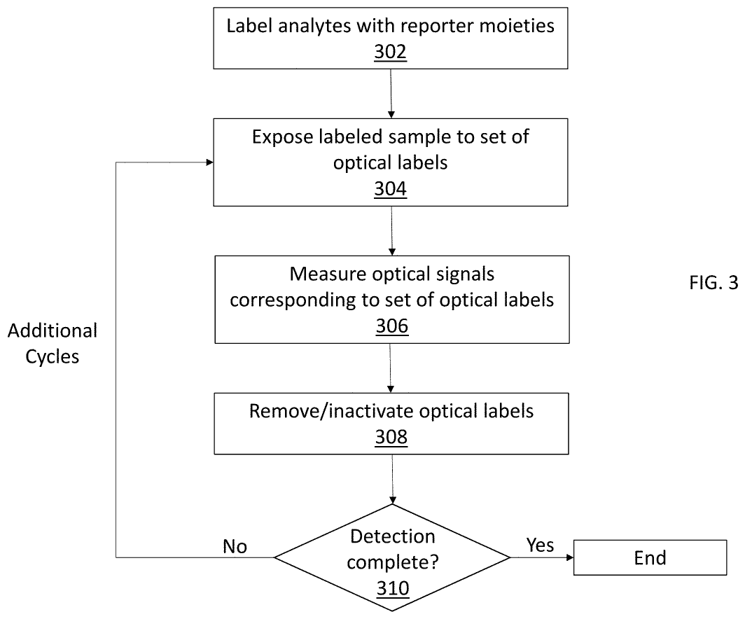

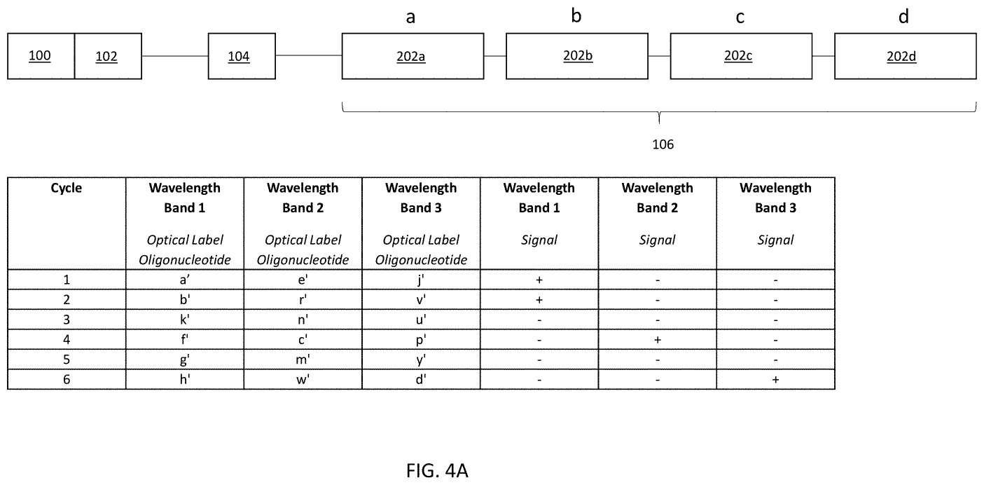

To detect analytes in a sample, one or more cycles of optical label hybridization and detection are generally performed. FIG. 3 is a flow chart that shows a set of example steps for detecting and quantifying multiple types of analytes in a sample. In step 302, the multiple analytes are conjugated to reporter moieties, with each different type of analyte conjugated to a different type of reporter moiety with a unique combination of label regions as discussed above.

Next, in step 304, the sample is exposed to a set of one or more optical labels. The set of optical labels typically includes between 1 and 8 different optical labels (e.g., two, three, or four different optical labels), but can generally include any number of optical labels.

In general, one or more of the optical labels introduced in step 304 can hybridize with one or more of the label regions in each of the different types of reporter moieties in the sample. To increase the efficiency with which different types of reporter moieties are identified in the sample (e.g., by reducing the number of detection cycles), the set of optical labels can be selected such that, for at least one (and more generally, more than one) of the different types of reporter moieties present in the sample, multiple different optical labels of the introduced set hybridize to different label regions of the reporter moiety/moieties, and generate optical signals. In this manner, multiple label regions of these reporter moieties can be identified in a single detection cycle, reducing the number of cycles required to fully elucidate all of the label regions associated with the reporter moieties. By selecting the optical label set in each cycle such that multiple different optical labels hybridize to different label regions of one or more of the different reporter moieties present in the sample, the number of detection cycles can be more efficiently utilized to identify the different reporter moieties, and therefore, to detect specific analytes in the sample.

Conversely, as a simplification of the above general optical label selection and utilization methodology, in some embodiments, the set of optical labels can optionally be selected such that in step 304, for each different type of analyte conjugated to a different type of reporter moiety, at most one of the optical labels in the set hybridizes to each different type of reporter moiety. In other words, in each detection cycle that includes step 304, at most one of the label regions of each different type of reporter moiety can be identified. As a consequence of this constraint on the selection of optical labels, each type of reporter moiety that includes D different label regions can be fully identified following a minimum of D detection cycles. As the number of different types of analytes conjugated to different types of reporter moieties increases, the number of detection cycles that are performed to fully identify each of the different types of reporter moieties generally increases, particularly if the number of different optical labels in each set remains constant.

While this choice of the optical label set may be less efficient than the more general methodology in which more than optical label hybridizes to one or more of the different types of reporter moieties in the sample as described above, this choice of optical label set can be useful in some circumstances. For example, in samples where optical labels may interact with one another (e.g., by fluorescence resonance excitation transfer, donor-acceptor quenching, or photoinduced electron transfer), it may be disadvantageous to introduce certain optical labels in a manner such that they hybridize to a common reporter moiety, as positioning the optical labels in such close proximity may promote such interactions, which disrupt successful detection of the label regions to which they hybridize by extinguishing, masking, or otherwise interfering with the signals generated by the optical labels. In such circumstances, by selecting the set of optical labels so that at most one optical label from the set hybridizes to each different type of reporter moiety in the sample, interactions between different types of optical labels can be reduced.

Next, in step 306, optical signals corresponding to the optical labels of the set are measured. In some embodiments, the optical signals are measured by obtaining one or more images (e.g., multispectral images) of the sample with the set of optical labels hybridized to complementary label regions of reporter moieties conjugated to the analytes of the sample. To obtain the one or more images, the sample is exposed to incident light, and signal radiation generated by the optical labels (e.g., fluorescence emission) is detected using an imaging detector such as a CCD array or CMOS-based array detector.

In general, each of the different optical labels that is introduced in step 304 generates signal radiation according to a different spectral distribution, and is therefore associated with a different detection channel. In practice, signal radiation in different detection channels can be detected in a variety of ways. In some embodiments, where each detection channel is well separated spectrally from the other detection channels, the signal radiation generated by each different type of optical label is relatively well isolated spectrally in a distinct detection channel. As such, signal radiation attributable to each of the different types of optical labels can readily be isolated and detected by spectral filtering (e.g., with a plurality of optical bandpass filters) and/or by using a spectrally resolving detector, such as a grating, prism, or other spectrally dispersive element in conjunction with a CCD array or CMOS-based array detector.

In certain embodiments, the spectral distributions of signal radiation generated by the different optical labels may overlap to a degree that is not insignificant, such that optical filtering and spectral dispersion methods alone are insufficient to isolate signal radiation generated by each of the different optical labels. Because the spectral distributions of the signal radiation are spectrally convolved to some extent, accurate detection of signals generated by each of the optical labels may therefore involve more complex spectral deconvolution techniques to accurately separate and assign measured signals to specific optical labels.

In such circumstances, sample images that include signal radiation from multiple different optical labels can optionally be decomposed into a set of images, in which each image in the set corresponds substantially only to signal radiation from one optical label. A variety of methods can be used to perform such decompositions, including for example spectral unmixing methods that involve performing an eigenvector decomposition of the measured optical signals into individual contributions from “pure” spectral components (e.g., contributions from each optical label).

In general, analytes are present in the sample at different spatial locations. As such, for each pixel in an image of the sample, any optical signals measured at that pixel will arise from optical labels hybridized to a reporter moiety that is linked to a capture moiety specifically bound to one analyte at a location in the sample corresponding to the pixel. In embodiments where, in a detection cycle, multiple optical labels hybridize to a reporter moiety, multiple optical signals are therefore generated and detected at a pixel corresponding to the reporter moiety. As described above, if the multiple optical signals are well separated spectrally, they can be resolved and detected by spectral filtering methods. Alternatively, if the multiple optical signals overlap spectrally, the measured signal at the pixel—which corresponds to a convolution of the multiple signals—can be decomposed into component signals (e.g., component images) that individually correspond to an optical signal generated substantially only by a single one of the optical labels.

Further, as described above, if the set of optical labels is selected so that, at most, one optical label in the set hybridizes to each reporter moiety 106 in the sample, then each pixel in the set of one or more images will include, at most, an optical signal corresponding to one of the optical labels in the set. In such circumstances, methods for spectrally decomposing the measured signals are generally not required to separate signals generated by multiple optical labels. However, such methods can still be used, for example, to compensate for the effects of autofluorescence and spectral contributions that may arise from other species present in the same that are not of interest.

It should also be noted that in each image of the sample, certain pixels may be “dark” (i.e., have zero or very low aggregate signal intensity). A dark pixel indicates that none of the optical labels hybridized to reporter moieties at a location in the sample corresponding to the dark pixel during the current detection cycle. If no optical signals are measured at a particular pixel among all images through all detection cycles, then none of the different types of analytes targeted by the capture moieties was present at the location in the sample corresponding to the dark pixel.

Step 306 yields a set of one or more images of the sample. Particular pixels at a common location in the set of images correspond to the same location in the sample, which is represented by the common pixel location in the images. Collectively, pixels across the set of images that correspond to a common pixel location are associated with optical signals generated by optical labels that hybridize at the corresponding location in the sample. Because the optical signals generated by each different type of optical label in a detection cycle are known, the presence of particular label regions at each pixel location in the images can be determined. After all detection cycles are complete, the particular set of label regions present at each pixel location can be used to determine the type of reporter moiety 106 present at the pixel location, and therefore, the analyte present at the pixel location, as described in further detail below.

Following step 306, the set of optical labels can be removed from the sample or inactivated in step 308. A variety of different methods can be used in step 308 for optical label removal or inactivation.

In some embodiments, optical labels that hybridize to label regions of reporter moieties can be readily removed by dehybridization. Dehybridization can be accomplished using various methods including, but not limited to: exposure to one or more chaotropic reagents; thermally-induced dehybridization via heating; toehold mediated strand displacement (TMSD); and enzymatic strand displacement using enzymes such as RNAse, DNAse.

In certain embodiments, portions of the optical labels can be removed from the reporter moieties, while other portions of the optical labels remain in the sample. For example, one or more reducing agents can be used to cleave covalent bonds that link a dye moiety to an oligonucleotide in an optical label. The cleaved dye moieties can then be washed from the sample. The oligonucleotides can optionally remain hybridized to label regions in the reporter moiety. A variety of different reducing agents can be used for this purpose. For example, tri(2-carboxyethyl)phosphine (TCEP) can be used to cleave dye moieties that are linked via disulfide bonds to oligonucleotides in optical labels.

In some embodiments, optical labels are not removed from the sample, but are instead inactivated so that they do not generate optical signals in subsequent detection cycles. Various methods can be used for inactivation of optical labels. For example, in certain embodiments, chemical bleaching of optical labels can be used to inactivate the labels.

Next, in step 310, if analysis of the analytes present in the sample is complete, then the workflow ends. However, if analysis is not complete, one or more additional cycles of steps 304, 306, and 308 are performed. In each additional cycle, a set of optical labels are introduced into the sample and associate with label regions of the reporter moieties in the sample. Optical signals corresponding to the set of optical labels are measured and optionally decomposed as described above, before the optical labels are optionally removed from the sample and/or deactivated.

The workflow shown in FIG. 3 can be repeated for any number of cycles to detect and quantify analytes 100 in the sample. In some embodiments, each set of optical labels that is used in a particular cycle is unique; that is, none of the optical labels are re-used in subsequent cycles. In this manner, individual label regions of each reporter moiety are detected only once through the entire workflow.

In certain embodiments, to perform error checking and/or to correct for binding and imaging errors such as incomplete hybridization and/or optical imaging aberrations, certain optical labels can be applied to the sample in more than one cycle in FIG. 3. By doing so, for example, optical signals measured in prior cycles can be verified, and optical signals that may have been absent in prior cycles (e.g., due to incomplete hybridization of the optical labels) can be measured in a later cycle.

It should also be noted that while in some embodiments each of the optical labels is introduced into the sample only once during the workflow shown in FIG. 3, optical labels that bind to different label regions of reporter moieties, but have the same dye, can be used in different cycles of the workflow. That is, provided that different optical labels in the same cycle do not include the same dye, measured signals can be unambiguously attributed to the different optical labels. This unambiguous assignment remains possible when different optical labels include the same dye, provided the optical labels are applied and corresponding signals measured during different cycles in FIG. 3.

In general, the workflow in FIG. 3 typically continues until a sufficient number of cycles have been performed to unambiguously identify each of the analytes. In some embodiments, the workflow continues until optical labels that bind or associate with each of the label regions of each of the reporter moieties 106 have been introduced, and signals attributable thereto have been measured.

In certain embodiments, depending upon the number of analytes, the number of different reporter moieties, and the number of label regions in each reporter moiety, it may be possible to unambiguously identify each of the analytes without introducing and measuring optical labels that bind or associate with every label region of every reporter moiety. For example, analytes in the sample can be conjugated to reporter moieties that include 6 label regions, but it may be possible to unambiguously identify certain analytes of interest by binding or associating optical labels to only a subset (e.g., 3 or 4) of the label regions of some or all of the reporter moieties. In this manner, one or more detection cycles may be omitted, saving the expense and time associated with the omitted cycles. This method may be applied, for example, to assays in which a limited number of analytes are of interest, while the generalized assay permits identification of a much larger number of different analytes. Foregoing detection cycles that only provide information about analytes that are not of interest, or that do not provide additional information that is useful for identifying analytes that are of interest, shortens the time associated with the assay, and may reduce the associated cost by limiting the number of different optical labels that are used.

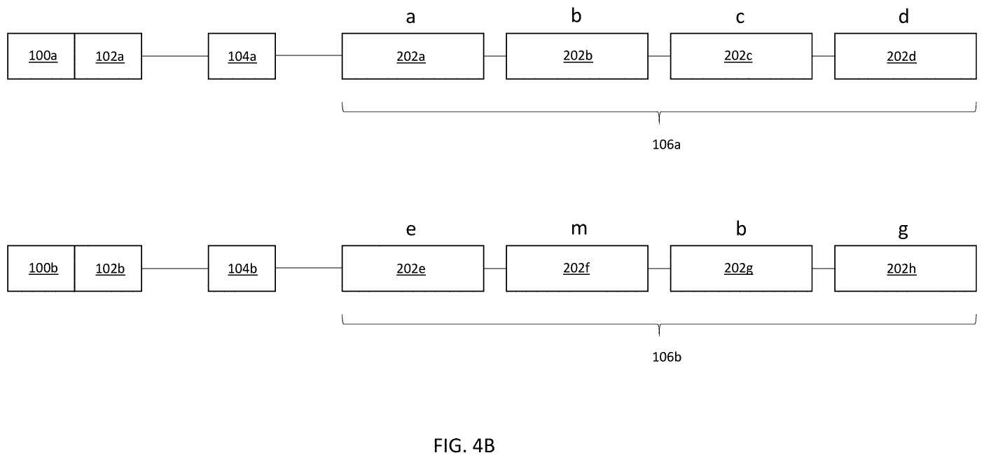

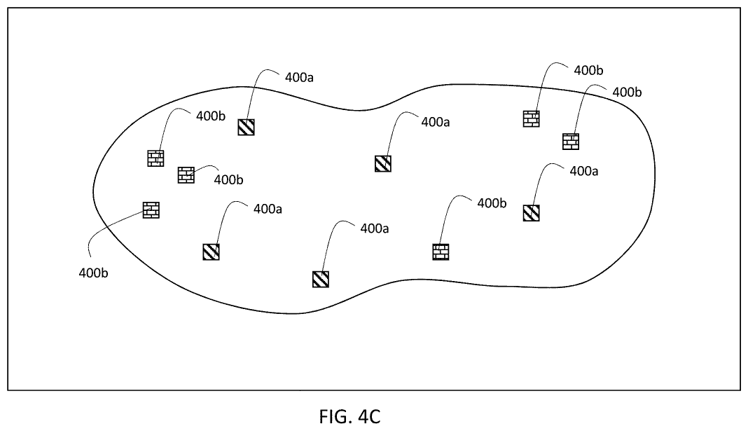

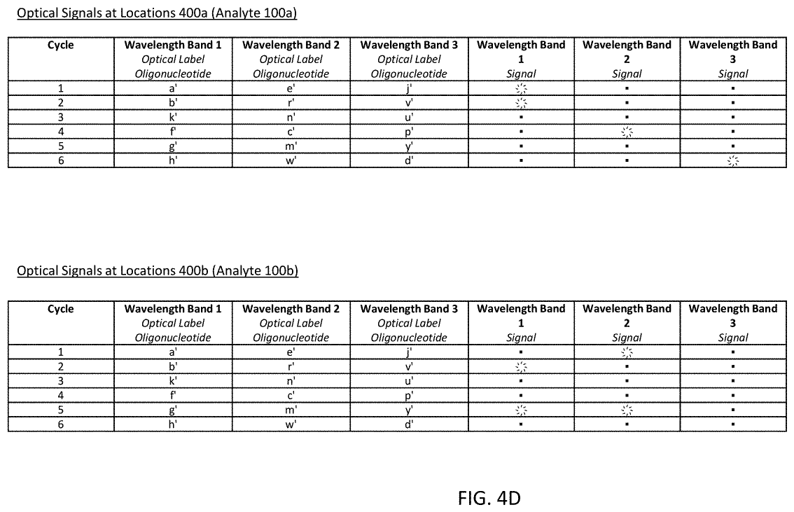

FIG. 4A is a schematic diagram showing an example illustrating the detection of an analyte 100 in sample 10 via multiple cycles of hybridization and dehybridization of oligonucleotide-based optical labels. Analyte 100 is bound to capture moiety 102, which is linked (through linker 104) to a reporter moiety that includes label regions 202a-d. Each one of label regions 202a-d includes a distinct oligonucleotide sequence, and the respective sequences are labeled a, b, c, and d for reference. For an oligonucleotide sequence designated x, a partially or fully complementary sequence that hybridizes to sequence x is designated x′. Thus, for example, sequence a′ is complementary to and hybridizes to sequence a, sequence b′ is complementary to and hybridizes to sequence b, and so forth.

To detect analyte 100, multiple detection cycles are performed as described above in connection with FIG. 3. In each cycle, one or more optical labels with oligonucleotide sequences are introduced into the sample. Optical labels with sequences that are at least partially complementary to sequences of label regions 202a-d hybridize to the corresponding label regions. Measurement of the signals generated by the hybridized optical labels reveals the presence of the label region at particular spatial locations in the sample. Co-location in the sample of signals attributable to each of the label regions in a particular reporter moiety reveals the presence the reporter moiety at that location in the sample, and therefore, the presence of analyte 100 to which the reporter moiety is conjugated.

In general, within each detection cycle, each different type of optical label includes a different oligonucleotide sequence, and generates a distinguishable optical signal. As such, within each cycle, following hybridization of one of more of the optical labels to complementary label regions of reporter moieties, the optical signals corresponding to the different types of optical labels can be measured and distinguished, identifying the different types of optical labels that have hybridized to complementary label regions of reporter moieties.

Optical signals generated by the different types of optical labels can be distinguishable in various ways. In some embodiments, for example, different types of optical labels include different dyes (e.g., fluorescent dyes) that emit light in different wavelength bands (i.e., bands that have different central wavelengths and/or different wavelengths of maximum emission intensity). If the wavelength bands are sufficiently separated spectrally, the optical signals can be distinguished from one another by spectral filtering and/or related techniques.

For optical signals that are not as well separated spectrally (e.g., when relatively higher numbers of different types of optical labels are used in a detection cycle), or that have non-Gaussian emission band shapes, a variety of computational techniques can be used to separate measured optical signals into component signals that individually correspond substantially to only one of the optical labels. For example, eigenvector decomposition methods such as spectral unmixing can be used to decompose multispectral images of a sample into a set of single-component images, each corresponding to spectral contributions from only one of the different types of optical labels.

To detect analyte 100 in sample 10, individual label regions of reporter moiety 106 in FIG. 4A are detected in different cycles of the detection sequence. For purposes of illustration, within each detection cycle in FIG. 4A, three different types of optical labels are introduced. Each of the different types of optical labels includes a different oligonucleotide, and a fluorescent moiety that generates an optical signal in one of three different wavelength bands. Optical signals in each of the three different wavelength bands are distinguishable from one another using the methods discussed above.

It should be noted that the three different wavelength bands can be the same or different in different detection cycles. In some embodiments, for example, three different types of optical labels are introduced in each detection cycle, and each of the different types of optical labels generates an optical signal in only one of three different wavelength bands, with the three different wavelength bands remaining the same among all cycles. In other words, the same set of three different wavelength bands is re-used in each detection cycle.

In certain embodiments, some or all of the wavelength bands in which the different types of optical labels generate optical signals are different from one cycle to the next. That is, some of wavelength bands may be re-used from one cycle to the next, or none of the wavelength bands may be re-used. In general, there are no constraints on the manner in which different wavelength bands can be re-used or not re-used, except that within a particular detection cycle, each of the different types of optical labels generates an optical signal that is distinguishable from optical cycles generated by other types of optical labels within the cycle.

It should also be used that the introduction of three different types of optical labels in each detection cycle in FIG. 4A is merely an example for illustrative purposes. More generally, any number of different types of optical labels can be introduced in each cycle. The number of different types of optical labels that can be introduced can be the same among all cycles, or can vary among the cycles. Further, in some embodiments, each type of optical label is introduced only once among all detection cycles, as shown in FIG. 4A. More generally, however, some or all of the optical labels can be introduced in more than one (e.g., two or more, three or more, four or more, five or more, or even more) of the detection cycles. Introducing one or more of the optical labels in multiple detection cycles can allow verification of previously-detected optical signals that have been attributed to specific optical labels, for example.

The table in FIG. 4A shows an example of several cycles of optical label hybridization and measurement to detect analyte 100 in the sample. In each cycle, a first type of optical label generates an optical signal in Wavelength Band 1, a second type of optical label generates an optical signal in Wavelength Band 2, and a third type of optical label generates an optical signal in Wavelength Band 3. It should be noted that Wavelength Bands 1, 2, and 3 may be the same from one cycle to the next, or some or all of the Wavelength Bands may differ between any two cycles. The indicators “1”, “2”, and “3” designate only that the Wavelength Bands are different, and optical signals generated in each Wavelength Bands are distinguishable to allow identification of the optical labels that generate the signals, and therefore, the complementary label regions to which they are hybridized.

In cycle 1, optical labels with respective sequences a′, e′, and j′ are introduced into the sample. The first type of optical label with sequence a′ generates an optical signal in Wavelength Band 1, the second type of optical label with sequence e′ generates an optical signal in Wavelength Band 2, and the third type of optical label with sequence j′ generates an optical signal in Wavelength Band 3. Only the optical label with sequence a′ hybridizes to reporter moiety 106 (i.e., to label region 202a with sequence a). Thus, in cycle 1, a positive signal (i.e., a non-zero optical signal) is measured at the location of analyte 100 in the sample in Wavelength Band 1. No positive signals are measured in either Wavelength Band 2 or Wavelength Band 3.

In cycle 2, optical labels with respective sequences b′, r′, and v′ are introduced. The first type of optical label with sequence b′ generates an optical signal in Wavelength Band 1, the second type of optical label with sequence r′ generates an optical signal in Wavelength Band 2, and the third type of optical label with sequence v′ generates an optical signal in Wavelength Band 3. Only the optical label with sequence b′ hybridizes to reporter moiety 106 (to label region 202b with sequence b). Therefore, in cycle 2, a positive signal is measured at the location of analyte 100 in the sample in Wavelength Band 1. No positive signals are measured in either Wavelength Band 2 or Wavelength Band 3.

The different types of optical labels introduced in cycles 3-6 are shown in FIG. 4A as well. Positive signals are measured at the location of analyte 100 in cycles 4 (in Wavelength Band 2 but not in Wavelength Bands 1 or 3) and 6 (in Wavelength Band 3 but not in Wavelength Bands 1 or 2). No positive signals are measured in any of the Wavelength Bands in cycles 3 or 5, as none of the optical labels in those cycles hybridizes to any of the label regions of reporter moiety 106. The combination of positive signals measured in cycles 1 (in Wavelength Band 1), 2 (in Wavelength Band 1), 4 (in Wavelength Band 2), and 6 (in Wavelength Band 3), directly identifies that sequences a, b, c, and d are present in reporter moiety 106, as those particular Wavelength Bands are assigned to optical labels with complementary oligonucleotide sequences a′, b′, c′ and d′ in cycles 1, 2, 4, and 6, respectively. As the combination of label regions a-b-c-d is specific for a capture moiety 102 that targets analyte 100, the presence of analyte 100 can be unambiguously determined in the sample. Further, the measured signal intensity is correlated with the amount of analyte 100 at each location in the sample, and therefore analyte 100 can be spatially quantified in the sample.

It should also be noted that the foregoing methods do not distinguish among different orderings of label regions within reporter moiety 106. In the example of FIG. 4A, by performing the 6 detection cycles shown, it can be determined that reporter moiety 106 includes label regions a, b, c, d. However, the specific order of label regions within reporter moiety 106 is not determined. Thus, the specific order of the label regions could be a-b-c-d, b-d-c-a, c-b-d-a, d-b-a-c, or any other ordered, non-repeating combination of the sequences a, b, c, and d.

In the example of FIG. 4A, in each set of optical labels that is introduced in each of the 6 detection cycles, at most one of the optical labels hybridizes to a label region of reporter moiety 106. However, as discussed above, more generally the sets of optical labels are selected such that in one or more of the detection cycles, multiple different optical labels can hybridize to individual reporter moieties 106, and generate optical signals that are detected and used to identify corresponding label regions in the reporter moieties. By selecting the sets of optical labels in this manner, the overall efficiency of the assay can be increased by making increased use of the available detection channels during each detection cycle.

The example shown in FIG. 4A is representative, and illustrates the detection of a single analyte 100. In general, however, a sample includes many different analytes, and the methods, compositions, and kits described herein are used to detect many different analytes. For example, in some embodiments, 10 or more (e.g., 20 or more, 30 or more, 40 or more, 50 or more, 60 or more, 70 or more, 80 or more, 100 or more, 120 or more, 140 or more, 160 or more, 200 or more, 300 or more, 400 or more, 500 or more, 700 or more, 1000 or more, 1500 or more, 2000 or more, 3000 or more, 5000 or more, 7000 or more, 10000 or more, 15000 or more, 20000 or more, 30000 or more, 40000 or more, 50000 or more, or even more) different analytes are detected in a sample using the methods, compositions, and/or kits described herein.

When a sample contains multiple different analytes, each different type of analyte selectively binds to a different type of probe that includes a capture moiety specific to that type of analyte, and a reporter moiety linked to the capture moiety. The multi-cycle detection methods described herein are used to detect the multiple different types of analytes. During each detection cycle, the different optical labels introduced in that cycle can hybridize to complementary label regions in the reporter moieties in the sample that are linked to different types of capture moieties. In general, in each cycle, the set of optical labels that are introduced can be selected so that multiple optical labels hybridize to at least some different types of reporter moieties, which ensures that individual detection cycles are efficiently used to elucidate the label regions—and therefore the reporter moieties—that are present in each location of the sample.

FIG. 4B shows a two probes that specifically bind to two different analytes in sample 10. Specifically, the first probe includes a capture moiety 102a that specifically binds to a first type of analyte 100a, and is linked through a linker 104a to a reporter moiety 106a that includes label regions 202a-202d with oligonucleotide sequences a, b, c, and d, respectively. The second probe includes a capture moiety 102b that specifically binds to a second type of analyte 100b, and is linked through a linker 104b to a reporter moiety 106b that includes label regions 202e-202h with oligonucleotide sequences e, m, b, and g, respectively.

FIG. 4C is a schematic image of sample 10. In sample 10, analyte 100a is present at locations 400a, while analyte 100b is present at locations 400b. It should be noted that only one type of analyte is present at each physical location in sample 10. In other words, two analyte molecules in sample 10 are not spatially coincident anywhere in sample 10. Instead, each analyte molecule is present at a distinct location, although particular pairs of analyte molecules may be very closely spaced within sample 10. The methods described herein can be used no matter how closely spaced two analyte molecules are in the sample.

FIG. 4D shows the measured optical signals from sample 10 at locations 400a and 400b over 6 detection cycles in tabular form. The upper table shows the measured optical signals at locations 400a (where analyte 100a is located in the sample). The lower table shows the measured optical signals at locations 400b (where analyte 100b is located in the sample). In each table, the sunburst character indicates that an optical signal was measured in a particular wavelength band, and the solid square character indicates that no optical signal was measured in a particular wavelength band.

As shown in column 1 of each table, 6 detection cycles were performed. Three optical labels were introduced in each detection cycle. The oligonucleotide sequences of each of the optical labels in each detection cycle, and the wavelength bands in which they generate optical signals, are shown in columns 2-4 of each table. Because the detection cycles were performed on the whole sample 10, columns 1-4 are identical in both tables.

The upper table indicates that positive optical signals were measured at locations (e.g., pixels) 400a in sample 10 in the following ordered pairs: (cycle 1, wavelength band 1), which corresponds to an optical label with oligonucleotide sequence a′; (cycle 2, wavelength band 1), which corresponds to an optical label with oligonucleotide sequence b′; (cycle 4, wavelength band 2), which corresponds to an optical label with oligonucleotide sequence c′; and (cycle 6, wavelength band 3), which corresponds to an optical label with oligonucleotide sequence d′. These positive optical signals are measured because reporter moiety 106a, which is exclusive present at locations 400a in the sample, includes label regions with oligonucleotide sequences a, b, c, and d.

The lower table indicates positive signals that were measured at locations (e.g., pixels) 400b in sample 10. Reporter moiety 106b is exclusively present at locations 400b, and contains label regions with oligonucleotide sequences e, m, b, and g. The positive signals measured at locations 400b in the sample are represented by the following ordered pairs: (cycle 1, wavelength band 2), which corresponds to an optical label with oligonucleotide sequence e′; (cycle 2, wavelength band 1), which corresponds to an optical label with oligonucleotide sequence b′; (cycle 5, wavelength band 1), which corresponds to an optical label with oligonucleotide sequence g′; and (cycle 5, wavelength band 2), which corresponds to an optical label with oligonucleotide sequence m′.

Following the 6 detection cycles shown in FIG. 4D, each of the image pixels corresponding to sample locations 400a is associated with positive signals for label regions a, b, c, and d. Because only reporter moiety 106a corresponds to that combination of label regions, the presence of analyte 100a at locations 400a can be unambiguously identified. Similarly, each of the image pixels corresponding to sample locations 400b is associated with positive signals for label regions e, b, g, and m. Only reporter moiety 106b corresponds to that combination of label regions, and so the presence of analyte 100b at locations 400b can be unambiguously identified.

As is apparent from the results of cycle 2, when different reporter moieties contain the same label region (reporter moieties 106a and 106b each contain a label region with oligonucleotide sequence b in FIG. 4B), optical signals are measured from optical labels that hybridize to each of the different reporter moieties in a detection cycle in which the complementary optical label (e.g., an optical label with oligonucleotide sequence b′) is introduced. However, the reporter moieties are associated with different analytes due to the specific nature of the interaction between the capture moieties and the analytes. In the present example, reporter moiety 106a is associated only with analyte 100a by virtue of the specific interaction between capture moiety 102a and analyte 100a, and reporter moiety 106b is associated only with analyte 100b by virtue of the specific interaction between capture moiety 102b and analyte 100b. As a result, the optical signals corresponding to reporter moiety 106a will be measured only at locations 400a, and the optical signals corresponding to reporter moiety 106b will be measured only at locations 400b, even though the optical signals are generated by the same optical label with oligonucleotide sequence b′.

Because analytes 100a and 100b are at spatially distinct locations in sample 10, the measured optical signals corresponding to the optical label with sequence b′ have different meanings. Signals measured at locations 400a correspond to reporter moiety 106a and analyte 100a, while signals measured at locations 400b correspond to reporter moiety 106b and analyte 100b.

In general, it is not known apriori where any of the analytes are located within the sample. Instead, the locations of particular types of analytes are determined from the accumulated optical signal measurements at each pixel location. Specifically, each location in a sample (which corresponds to a particular pixel in the sample images that are obtained for detection of optical signals) is associated with a set of measured optical signals generated by optical labels during the multiple cycles of detection. At each location/pixel, the set of measured optical signals is associated with certain optical labels that were introduced during the detection cycles. Because the oligonucleotide sequences of the optical labels were known, the set of measured optical signals at each location/pixel is associated with a set of label regions at that location/pixel.

Each different type of reporter moiety contains a unique combination of label regions, without regard to ordering of the label regions. Consequently, the set of label regions at a particular location/pixel uniquely associates that location/pixel with a particular reporter moiety, and therefore, a particular analyte at that location/pixel. In this manner, at each location/pixel within the sample, the presence of any one of the analytes can be detected. Referring again to the example in FIGS. 4B-4D above, the co-location of label regions a, b, c, and d at locations 400a in sample 10 identifies analyte 100a at locations 400a. Similarly, the co-location of label regions e, m, b, and g at locations 400b in sample 10 identifies analyte 100b at locations 400b.

For locations/pixels at which no set of label regions can be determined (i.e., no optical signals are measured), no analyte is located at those locations/pixels. For locations/pixels at which an incomplete or erroneous set of label regions is determined, the presence or absence of an analyte and the nature of the analyte at those locations/pixels is indeterminate if the determined set of label regions cannot be corrected.

As discussed previously, in some embodiments, it can be advantageous to introduce one or more optical labels in two or more of the detection cycles in a multi-cycle detection procedure. For example, referring again to the example of FIG. 4A, after cycle 6, an additional detection cycle 7 can be performed in which one or more of the optical labels previously introduced is introduced again. By way of example only, consider a detection cycle in which optical labels with oligonucleotide sequences a′ (associated with wavelength band 1), s′ (associated with wavelength band 2), and q′ (associated with wavelength band 3) are introduced. The optical labels corresponding to sequences s′ and q′ will not hybridize to reporter moiety 106, and therefore generate no measured signal in any of the wavelength bands. The optical labels corresponding to sequence a′ will hybridize again to reporter moiety 106, and generate optical signals in wavelength band 1.

The signals are detected, as described above, by obtaining one or more images of the sample. Nominally, the spatial distribution of signals corresponding to the optical label with sequence a′ should be the same as the spatial distribution of signals corresponding to the optical label with sequence a′ in cycle 1. Accordingly, the measured optical signals generated by the optical labels with sequence a′ at each of the locations/pixels in cycle 7 can be used as a verification of the optical signals corresponding to optical labels with sequence a′ that were measured in cycle 1. Discrepancies may be corrected, or pixels at which discrepancies are observed can be marked and omitted from further analysis.

Thus, while introducing optical labels in more than one detection cycle can increase the time over which the assay is performed, doing so can also provide verification of previously measured optical signals, which can be an important consideration when the sample is of poor quality and/or certain analytes are not easily or reproducibly bound to capture moieties. It should be noted that the introduction of optical labels in more than one detection cycle is advantageous when optical labels are dehybridized following measurement of optical signals. If the optical labels are instead deactivated or only partially removed, later re-introduction of the same optical labels may be more difficult.

### Selection of Reporter Codes

In the foregoing discussion, the primary constraint that has been applied to the label regions of the reporter moiety 106 is that the combination of label regions in each reporter moiety is unique. For reporter moieties 106 that include oligonucleotide label regions, this constraint specifies that the oligonucleotide sequences of the label regions in each type of reporter moiety (i.e., reporter moieties that are used to identify a particular type of analyte 100 through selective binding of capture moiety 102) is not repeated among reporter moieties that are used to identify other types of analytes.

In practice, other constraints can be applied to the label regions of reporter moiety 106 to further ensure that particular types of reporter moieties and the analytes to which they are selectively bound are detected, and to correct for errors that can occur during binding (e.g., hybridization) of the optical labels to the label regions and imaging of the bound or associated optical labels. Errors that can occur during binding can include, but are not limited to: incomplete hybridization of optical labels to corresponding label regions in reporter moieties; inadvertent dehybridization of optical labels during, or prior to, measurement of optical signals; cross-hybridization of optical labels to multiple different label regions in reporter moieties; and incomplete removal of optical labels at the end of a cycle. Errors that can occur during imaging include, but are not limited to: obscuration of optical signals generated by optical labels bound to reporter moieties (e.g., obscuration by sample features or conformational changes of the reporter moieties and/or optical labels); inadvertent quenching of optical signals generated by optical labels by other sample features; and loss of resolution due to non-specific localization of optical labels.

In general, constraints that are used to configure the label regions of reporter moiety 106 can be applied to correct for a number of different types of errors that can arise when labeling analytes and measuring corresponding optical signals from the labeled analytes. These include, for example, reducing or eliminating erroneous hybridization of optical labels to label regions of reporter moiety 106. Hybridization of an optical label to a label region to which it is not intended to correspond, but nonetheless a certain extent of inadvertent complementarity exists between the sequences of the label region and the optical label, is generally not corrected in this manner. However, erroneous spatial location of an optical label in the vicinity of a label region of reporter moiety 106 that occurs once in anomalous fashion can be determined and corrected, as can the absence of an optical label in the vicinity of a label region of reporter moiety 106 that occurs once, when the optical label and label region would have been expected to hybridize.

Errors that can be corrected can also include incorrect assignment of individual regions (which may correspond to voxels in a sample) that arise when different optical labels generate optical signals that are spectrally similar. When measured signals from different optical labels are sufficiently similar, it can be challenging to assign spatial regions associated with the signals to the correct labeled analyte. Such errors can be reduced or minimized, for example, by distributing optical labels among multiple fluorescence channels (i.e., selecting the optical labels such that they have fluorescence emission spectra that are sufficiently different), such that optical signals measured from the different optical labels can be readily distinguished.

Additional errors that can be corrected through selection of appropriate combinations of optical labels, label regions, and the use of multi-cycle detection methodologies include the absence of detectable optical signals when hybridization should occur between a label region of reporter moiety 106 and an optical label. Under ordinary circumstances, an optical label that is sufficiently complementary to a label region of reporter moiety 106 hybridizes to the label region, and an optical signal generated by the optical label can be detected. When hybridization does not occur, however, due to factors such as stereochemical constraints and/or inadvertent dehybridization, the expected optical signal due to the optical label may not be measured, resulting in the absence of a measured optical signal that would otherwise be expected and used for identification of a labeled analyte. By appropriate selection of optical labels, label regions in reporter moiety 106, and the use of multi-cycle detection methodologies, the absence of expected measured optical signals during detection can be corrected, ensuring that labeled analytes in the sample can still be identified.

To provide for correction of errors during identification of multiple analytes in a sample labeled with different reporter moieties 106, the individual label regions of the different reporter moieties are selected according to certain constraints, and the optical labels that are used in the multi-cycle detection methodology described above in connection with FIG. 3 are also selected appropriately to ensure that individual labeled analytes can still be distinguished from another, even when certain types of detection errors occur during the multi-cycle detection methodology.

To further describe the selection of label regions and optical labels in connection with the multi-cycle detection methodology, the following terminology is used. In general, each reporter moiety 106 includes M different label regions. For M=4, as shown in FIG. 4, the label regions are 202a-202d. More generally, however, a reporter moiety 106 can include any number M of label regions, such that M can be 2 or more (e.g., 3 or more, 4 or more, 5 or more, 6 or more, 7 or more, 8 or more, 9 or more, 10 or more, or even more).

Typically, each different type of analyte in the sample that is analyzed is labeled with a different type of reporter moiety 106, i.e., a reporter moiety 106 with a different combination of label regions. In some embodiments, each of the different types of reporter moieties includes the same number M of label regions. In certain embodiments, different types of analytes are labeled with different types of reporter moieties 106, and among the different types of reporter moieties, one or more have different numbers of label regions than the other types of reporter moieties. For example, among the different types of reporter moieties, there can be one or more (e.g., 2 or more, 3 or more, 4 or more, 5 or more, 6 or more, 7 or more, 8 or more, 9 or more, 10 or more, or even more) groups of different types of reporter moieties, each group having a different number of label regions. Each such group can include one or more (e.g., 2 or more, 3 or more, 4 or more, 5 or more, 6 or more, 7 or more, 8 or more, 9 or more, 10 or more, or even more) label regions.

Each label region consists of multiple nucleotide bases that form a nucleotide sequence. In some embodiments, each of the M label regions within a reporter moiety have the same number of nucleotide bases. In certain embodiments, one or more of the M label regions consist of a first number of nucleotide bases, and one or more of the M label regions consist of a second number of nucleotide bases that is different from the first number of nucleotide bases. In general, any of the M label regions within a reporter moiety can include any number of nucleotide bases.

Among multiple reporter moieties used to label different analytes in a sample, in certain embodiments the M label regions in a first reporter moiety used to label a first analyte can each have the same number of nucleotide bases as the M label regions in a second reporter moiety used to label a second analyte that is different from the first analyte. More generally, however, the number of nucleotides in one or more of the label regions of a first reporter moiety can differ from the number of nucleotides in one or corresponding label regions of a second reporter moiety. Each label region in any of the reporter moieties described herein can independently include 2 or more (e.g., 3 or more, 4 or more, 5 or more, 10 or more, 15 or more, 20 or more, 30 or more, 40 or more, 50 or more, 60 or more, 70 or more, 80 or more, 90 or more, 100 or more, 120 or more, 140 or more, 160 or more, 180 or more, 200 or more, or even more) nucleotide bases. The number of nucleotide bases in any label region can be selected, for example, to control the kinetics, binding strength, and stringency of hybridization between the label region and optical labels.

The M label regions in each reporter moiety 106 form a reporter code C. In general, each analyte in the sample is labeled with a different reporter moiety 106, i.e., a reporter moiety 106 that contains a unique combination of M label regions forming a reporter code C. Thus, to detect T different analytes in a sample, a pool of reporter moieties with T unique reporter codes C is used. In particular, the sample is exposed to a composition that includes T unique reporter moieties 106, each of which is linked (e.g., through a linker 104) to a corresponding capture agent 102 that specifically binds a different one of the analytes. All reporter moieties with the same type of reporter code C are linked to the same type of capture agent, so that multiple analytes of the same type in the sample are labeled with the same type of reporter moiety having the same reporter code C. In this manner, each different type of analyte in the sample is labeled with a unique reporter moiety having a unique reporter code C. Detecting each of the reporter codes C therefore allows each of the different types of analytes to be identified in the sample, and determining quantities of each of the different reporter codes C allows the different types of analytes to be quantified in the sample.

In general, the number T of unique reporter codes, which may be the same as the number of unique reporter moieties 106 in a composition to which the sample is exposed, and may also be the same as the number of unique analytes that are detected, can be selected as desired. For example, T can be 2 or more (e.g., 3 or more, 4 or more, 5 or more, 10 or more, 15 or more, 20 or more, 25 or more, 30 or more, 35 or more, 40 or more, 45 or more, 50 or more, 60 or more, 70 or more, 80 or more, 90 or more, 100 or more, 200 or more, 300 or more, 400 or more, 500 or more, 600 or more, 800 or more, 1000 or more, 1500 or more, 2000 or more, 2500 or more, 3000 or more, 3500 or more, 4000 or more, 5000 or more, 6000 or more, 7000 or more, 8000 or more, 9000 or more, 10000 or more, 15000 or more, 20000 or more, 30000 or more, 40000 or more, 50000 or more, or even more).

To understand the nature of errors during hybridization of optical labels and detection of corresponding signals to identify analytes, consider a pool of N different oligonucleotide sequences. For purposes of illustration only, N can be assumed to be 26, with each of the oligonucleotide sequences being designated with a single-letter identifier from “a” to “z”. It should be noted that this value of N and the single-letter oligonucleotide sequences identifiers are used only by way of explanation, and in general, N can have any value, and the oligonucleotide sequences can be designated in any manner.

Consider further a particular reporter code C* having four label regions with oligonucleotide sequences a-b-c-d. Reporter code C* is part of reporter moiety 106, which is linked to capture moiety 102. Capture moiety 102, in turn, specifically binds to a particular analyte in the sample at spatial location L in the sample.

During multiple detection cycles, to detect the presence of reporter code C*, optical labels with complementary oligonucleotide sequences a′, b′, c′, and d′ are sequentially hybridized in some order to corresponding label regions of reporter code C*. Through multiple cycles of hybridization and detection, optical signals corresponding to hybridized optical labels with the sequences a′, b′, c′, and d′ are detected at location L in the sample. Based on this information, it can be determined that reporter code C* is presented at location L. Further, because reporter code C* is conjugated to a capture moiety 102 that specifically binds to a particular analyte Y, the presence of that analyte Y at location L can be identified.

In some embodiments, the T unique reporter codes C are selected such that each of the codes is distinguishable when a single drop error occurs during hybridization and detection of optical labels. In the foregoing example, optical signals corresponding to optical labels with oligonucleotide sequences a′, b′, c′, and d′ are expected to be measured at location L. A single drop error occurs when an optical signal corresponding to one of these optical labels is not measured at location L. Any one of the expected optical signals can be the missing signal. For example, measured optical signals at location L can correspond to optical labels having sequences a′, b′, and d′, and therefore the reporter code C* based on the measured optical signals at location L is a-b-d.

Under these circumstances, reporter code C* determined from multiple cycles of hybridization of optical labels and subsequent detection of emitted light from the hybridized labels is in error, as it only contains three label regions a, b, d, rather than four label regions. Without any restriction on the selection of the T unique reporter codes C that are conjugated to capture moieties within a pool of probes, it may not be possible to determine which analyte is located at location L in the sample. This ambiguity arises because it may not be possible to determine which additional label region (i.e., from among a-z) was present within reporter code C*, and was not detected.

To ensure that detected reporter codes C with a single drop error can still be distinguished from other reporter codes C in a composition of probes, the reporter codes can be selected according to particular constraints. In general, the composition of probes includes different types of probes, where each different type of probe includes a capture moiety 102 linked to a different type of reporter moiety 106 that contains a different type of reporter code C. To ensure that reporter codes with single drop errors can be distinguished from other reporter probes, each type of reporter code C is selected such that it differs from each of the other types of reporter codes C in the composition by at least two label regions, irrespective of the relative positions of the label regions in the reporter codes.

Thus, for example, if a composition contains one type of reporter code a-b-c-d present in one type of reporter moiety 106 that is linked to a capture moiety 102 that selectively binds a first type of analyte, then the composition does not contain any of the following examples of reporter codes in any different type of reporter moiety 106 linked to a capture moiety 102 that selectively binds a second type of analyte different from the first type of analyte: a-b-c-e (because this code differs from a-b-c-d by only one label region), d-c-b-f (because this code differs from a-b-c-d by only one label region), b-g-d-a (because this code differs from a-b-c-d by only one label region), and c-a-h-b (because this code differs from a-b-c-d by only one label region). Among the foregoing examples, the order of the label regions does not matter for purposes of determining the differences between reporter codes. Thus, d-c-b-f differs from a-b-c-d by only one label region (e.g., f has replaced a), even though the relative ordering of label regions b, c, and d differs between the codes.

Because the relative ordering of the label regions does not matter, the composition also does not contain any of the following examples of reporter codes in any different type of reporter moiety: a-d-b-c, c-d-a-b, and b-a-d-c. Each of these reporter codes does not differ from a-b-c-d at all, because the relative ordering of the label regions does not matter. In some embodiments, codes such as this—which differ only from a-b-c-d in the relative ordering of the label regions—are not present at all in the composition. In certain embodiments, codes such as this may be present in the composition, but only as part of a reporter moiety 106 that is linked to a capture moiety that selectively binds the first type of analyte.

It should be noted that the foregoing examples of reporter codes are not exhaustive, and the constraint discussed above excludes other reporter codes C from the reporter moieties 106 of the composition as well.

With the above constraint applied to the selection of reporter codes C present in different types of reporter moieties of different types of probes in the composition, detected reporter codes that result from a single-drop error can still be distinguished. For example, if a reporter code a-b-c-d is detected as a-b-d due to a single-drop error, then the detected code a-b-d can still be distinguished from other reporter codes of other types of probes, because the other reporter codes of other types of probes in the composition will not have the combination of a, b, and d label regions, since each reporter code differs from all other reporter codes linked to different types of capture moieties by at least two label regions. In other words, a detected reporter code a-b-d that contains a single drop error can still be distinguished from reporter codes such as a-b-e-f, d-b-r-s, b-a-f-g, and g-h-c-d.

In some embodiments, the T unique reporter codes C are selected such that each of the codes is distinguishable when a single add error occurs during hybridization and detection of optical labels. Continuing the foregoing discussion, optical signals corresponding to optical labels with oligonucleotide sequences a′, b′, c′, and d′ are expected to be measured at location L. A single add error occurs when an optical signal corresponding to an additional optical label is measured at location L. The additional optical signal can correspond to any one of the optical labels. For example, measured optical signals at location L can correspond to optical labels having sequences a′, b′, c′, d′, and e′, and therefore the reporter code C* based on the measured optical signals at location L is a-b-c-d-e.

To ensure that detected reporter codes C with a single add error can still be distinguished from other reporter codes C in a composition of probes, each type of reporter code C is selected such that it differs from each of the other types of reporter codes C in the composition by at least two label regions, irrespective of the relative positions of the label regions in the reporter codes.

Referring to the example above, suppose in addition to reporter code a-b-c-d as part of a first probe, which is erroneously detected as a-b-c-d-e, the composition also contains reporter code a-b-e-f as part of a second probe, which differs from reporter code a-b-c-d by two label regions (i.e., the third and fourth label regions). If reporter code a-b-e-f is correctly detected, then the second probe can be distinguished from the first probe based on label region f (i.e., based on a measured signal from an optical label having an oligonucleotide sequence f). In similar fashion, when reporter codes are present in probes of a composition according to the above constraint, each different type of probe—and the different analytes to which they selectively bind—can be distinguished, even when reporter codes are detected with single add errors.

In some embodiments, the T unique reporter codes C are selected such that the occurrence of a single replacement error during hybridization and detection of optical labels can be determined. Returning again to the discussion above, optical signals corresponding to optical labels with oligonucleotide sequences a′, b′, c′, and d′ are expected to be measured at location L. A single replacement error occurs when an optical signal corresponding to an unexpected optical label is measured at location L, in place of an expected optical label signal. The replacement optical signal can be detected in place of any one of the optical labels. For example, measured optical signals at location L can correspond to optical labels having sequences a′, b′, c′, and e′, and therefore the reporter code C* based on the measured optical signals at location L includes sequences a, b, c, and e, instead of sequences a, b, c, and d.

However, while a reporter code C corresponding to a combination of sequences a, b, c, and e can be recognized as invalid, the reporter code cannot necessarily be corrected, because it is not known which of the sequences a, b, c, and e was detected erroneously. That is, any one of sequences a, b, c, or e may have been the sequence that was detected in error. Thus, while the determined reporter code C that includes sequences a, b, c, and e can be identified as invalid, it may not be possible to determine the correct reporter code without additional information. Accordingly, detection signals corresponding to such reporter codes can simply be eliminated from further consideration on the grounds that they are erroneous.

To ensure that detected reporter codes C with a single replacement error can be identified as erroneous, each type of reporter code C is selected such that it differs from each of the other types of reporter codes C in the composition by at least two label regions, irrespective of the relative positions of the label regions in the reporter codes.

In addition to single add, single drop, and single replacement errors, other types of detection errors specific to reporter codes can also occur. One such example is a double drop error, in which a reporter moiety 106 contains Q label regions, but only (Q−2) label regions are detected. For example, reporter moiety 106 can include 4 label regions a-b-c-d forming a reporter code C, but during detection, only label regions a and b are detected. Thus, the detected reporter code is a-b.

Another example is a double add error, in which a reporter moiety 106 contains Q label regions, but (Q+2) label regions are detected. As an example, reporter moiety 106 includes label regions a-b-c-d forming reporter code C, but during detection, the reporter code is determined to be a-b-c-d-e-f.

In some embodiments, double add and/or double drop errors can be identified, but cannot be corrected without additional information. Consequently, as discussed above in connection with single replacement errors, corresponding detection signals can simply be eliminated from further consideration, on the grounds that they are erroneous.

In certain embodiments, double add and/or double drop errors can be corrected by applying constraints to the selection of reporter codes C as described above. For example, to be able to distinguish reporter codes of two reporter moieties 106 that are linked to two different types of capture agents 102, the reporter codes C in a composition can be selected according to the constraint that each type of reporter code C is selected such that it differs from each of the other types of reporter codes C in the composition by at least three label regions, irrespective of the relative positions of the label regions in the reporter codes. When reporter moieties in a composition differ by at least three label regions, reporter moieties that are subject to erroneous detection with a double add or double drop error can still be distinguished from other reporter moieties in the composition.

In some embodiments, other constraints can be applied to the selection of reporter codes C to ensure that different types of reporter moieties can be distinguished from one another during hybridization and detection of optical labels. For example, reporter codes C can be selected such that each code does not contain any duplicate oligonucleotide sequence. In other words, each reporter moiety 106 contains Q label regions, and each of the Q label regions is different in the reporter moiety.

The number of available reporter codes C depends on the constraints applied to the selection of the codes. To illustrate the relevant factors, consider an example where each reporter moiety includes Q=4 label regions (i.e., each reporter code C contains 4 oligonucleotide sequences). A group of reporter codes C are selected for use in detecting analytes in a sample such that among the group of codes, each code differs from each of the other codes in the group by at least two oligonucleotides (e.g., each type of reporter moiety to which the reporter code corresponds differs from each of the other types of reporter moieties by at least two label regions, without regard for the ordering of the label regions). Furthermore, each code does not contain any repeating oligonucleotide (e.g., each type of reporter moiety contains 4 distinct label regions, none of which repeat within the reporter moiety).

One can compute a set of suitable codes using a pool of N nucleotide sequences taken Q at a time, by first assembling a list of all possible codes; accepting the first code; then considering each remaining possible code in turn and either accepting it if it differs from the other accepted codes by at least 2 oligonucleotides or rejecting it if it differs by only 1 nucleotide from the other accepted codes. This continues until all possible codes have been considered. The result is a set of accepted codes that all differ from one another by at least two nucleotides. This can be done manually or using a computer program.

If each reporter code C is contained within a different type of reporter moiety 106 that is linked to a different type of capture moiety 102 that selectively binds to a different analyte, then the number of distinct analytes that can be detected, absent other considerations, is given by Tₘₐₓ. Examples of the number of distinct analytes that can be detected for different values of N and Q are shown in Table 1. These illustrate that a large number of analytes can be detected with a relatively small number of nucleotides N, where each moiety contains a relatively small number of label regions Q.

TABLE 1

| N | Q | Tₘₐₓ |
|---|---|---|
| 18 | 4 | 140 |
| 27 | 4 | 594 |
| 36 | 4 | 1320 |
| 18 | 5 | 336 |
| 27 | 5 | 2474 |
| 18 | 6 | 672 |

### Calibration of Detected Optical Signals

In some embodiments, additional calibration and error checking steps can be performed during multi-cycle hybridization and detection of optical labels. For example, referring again to FIGS. 3 and 4A-4D, to decode a reporter moiety 106, optical labels that hybridize to complementary label regions of the reporter moiety 106 are typically introduced once during a multi-cycle hybridization and detection procedure. A single exposure of all reporter moieties 106 in a sample to a particular optical label is generally sufficient to ensure that all complementary label regions among all reporter moieties in the sample hybridize to the particular optical label, and optical signals generated by the optical label are detected.

However, in some embodiments, one or more of the optical labels are hybridized to reporter moieties 106 in more than one cycle of the multi-cycle detection procedure. Repeating the exposure to one or more optical labels can be performed as a verification of measured detection signals arising from the optical labels. For example, an initial hybridization of a particular optical label to reporter moieties in a sample yields a spatial distribution of optical signals corresponding to the optical label. By repeating exposure to the same optical label in a later cycle, the measured spatial distribution of optical signals (i.e., obtained from an image of the sample) can be verified and, if necessary, corrected based on the second measured spatial distribution of optical signals obtained after the repeated exposure. Verification can be used both to ensure that spurious optical signals were not measured initially, and that optical signals that should have been measured initially were not undetected. By verifying the measured optical signals in such a manner, measurements of the spatial distributions of particular analytes can be confirmed. Furthermore, quantitative measurements of particular analytes, which are obtained by integrating spatial distributions of the analytes, can also be verified.

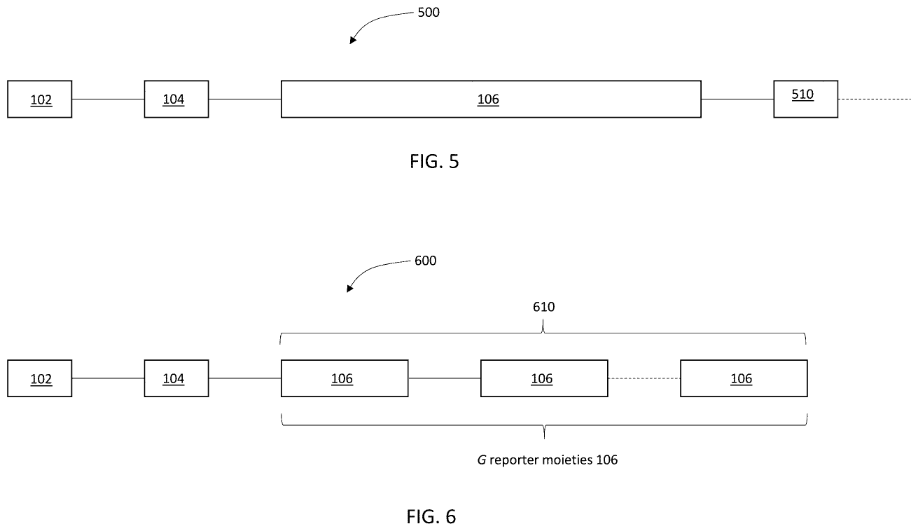

In certain embodiments, probes that include reporter moieties can also include binding regions for optical labels that are not part of reporter moieties, but instead are used to perform standardization or calibration functions. FIG. 5 is a schematic diagram showing an embodiment of a probe 500 that includes many of the features of the probes discussed above. In addition to a reporter moiety 106 that includes Q label regions, probe 500 includes one or more verification regions 510. Each of the verification regions 510 generally includes an oligonucleotide sequence that can have some or all of the same properties as the label regions discussed previously.

The sequences of each of the verification regions 510 are generally selected such that they are not complementary to any of the sequences of the optical labels. As such, during hybridization and detection of optical labels, none of the optical labels hybridizes to the verification regions of probe 500, and no optical signals corresponding to optical labels hybridized to the verification regions are measured.

However, the procedure shown in FIG. 3 can be modified to include one or more standardization steps in which a standardization optical label, which generates an optical signal that is different from the optical signals generated by the optical labels that hybridize to the label regions of the reporter moieties, is introduced into the sample and hybridizes to one of the verification regions of the probe. The standardization optical label generates a standardization optical signal which is detected in the same manner discussed previously.

In general, probe 500 can include one or more (e.g., two or more, three or more, four or more, five or more, or even more) verification regions 510. Each of the verification regions in a probe 500 can be different (e.g., can have a different oligonucleotide sequences), or alternatively, one or more of the verification regions 510 can be the same as another of the verification regions in probe 500.

Among a plurality of probes 500 introduced into a sample, and which members of the plurality of probes bind to different analytes in the sample, in some embodiments, each member of the plurality of probes can include the same set of one or more verification regions 510. Alternatively, in certain embodiments, some or all of the verification regions 510 among the plurality of probes can differ. For example, where the plurality of probes includes probes that selectively bind to multiple different types of analytes (e.g., multiple different nucleic acids in a sample), each of the probes can have a first verification region that is common to all probes, and a second verification region that differs among some of the probes. The sequence of the second verification region can depend, for example, on a particular class or grouping into which the analyte that is selectively bound by the probe falls. As one example, the sequence of the second verification region can be selected based on the origin of the analyte that is bound by the probe.

In this manner, measured optical signals corresponding to particular groups or classes of analytes can be reliably distinguished from other optical signals in a multi-analyte assay by measuring signals corresponding to a standardization optical label that hybridizes to a standardization region of the probes. Further, and in particular by measuring optical signals arising from a standardization optical label that hybridizes to a standardization region that is present in all probes, optical signals measured in different portions of a sample can be corrected relative to one another. That is, the optical signal arising from the standardization optical label functions as a common reference optical signal across the sample, in which variations in the signal can be presumed to arise from sample inhomogeneity, variance in detection sensitivity, and other factors that may yield variations in imaging across a spatial field of view. Measured optical signals arising from optical labels that hybridize to the label regions of reporter moieties 106 can be corrected for such variations, using the optical signal from the standardization optical label as a reference signal. A wide variety of such corrections are possible, including but not limited to dividing optical signals arising from reporter moieties by the corresponding standardization optical label signal (i.e., a spatially-resolved correction), normalizing or scaling optical signals arising from reporter moieties to yield signals that deviate reproducibly from the reference standardization optical signal, and using the standardization optical signal to establish thresholds for positive and negative signal detection among the measured optical signals arising from optical labels that hybridize to label regions of the reporter moieties.

The foregoing discussion has focused on probes for specific analytes that contain a reporter moiety 106. In some embodiments, probes can contain multiple reporter moieties 106 to effectively amplify measured signals corresponding to specific label regions that are used to identify particular analytes.

FIG. 6 shows a schematic diagram of a probe 600 that includes many features that are similar to probe 500 in FIG. 5. Probe 600 includes a detection moiety 610 that includes G reporter moieties 106. In general, each of the reporter moieties contains the same unique combination of label regions that is uniquely associated with the reporter moiety, and with the analyte that selectively binds to capture moiety 102. In some embodiments, each of the G reporter moieties 106 are the same. In certain embodiments, some of the reporter moieties differ from one another, but each reporter moiety still includes the same unique combination of label regions that is associated with the analyte that binds to capture moiety 102.

Detection moiety 610 can generally include any number G of reporter moieties 106. In some embodiments, for example, G is 1 or more (e.g., 2 or more, 3 or more, 5 or more, 7 or more, 10 or more, 15 or more, 20 or more, 25 or more, 30 or more, 35 or more, 40 or more, 50 or more, 60 or more, 70 or more, 100 or more, 120 or more, 150 or more, 200 or more, 300 or more, 400 or more, 500 or more, 700 or more, 1000 or more, 1200 or more, 1400 or more, 1600 or more, 1800 or more, 2000 or more, 2500 or more, 3000 or more, 4000 or more, 5000 or more, 7000 or more, 10000 or more, or even more).

Each of the reporter moieties 106 in detection moiety 610 can generally and independently have any of the properties and features described above in connection with reporter moieties. Further, detection moiety 610 can also include any of the other species, moieties, and sequences described herein, such as, but not limited to, one or more verification regions. Capture moieties 102 and linkers 104 present in probe 600 can generally and independently have any of the properties and features described herein in connection with capture moieties and linkers.

Probe 600, which includes multiple reporter moieties 106, can be fabricated using various methods. In some embodiments, for example, probe 600 can be formed by performing an extension reaction, such as rolling circle amplification (RCA).

In certain embodiments, probe 600 can be performed by successively concatenating reporter moieties 106 in sequential fashion. Each reporter moiety can include a first reactive end that forms a covalent bond with an activated second end of the reporter moiety. The second end of the terminal reporter moiety 106 in detection moiety 610 can be activated (e.g., by photolabilizing a protecting group at the second end of the terminal reporter moiety), and a new reporter moiety introduced. A covalent bond is formed between the activated second end of the terminal reporter moiety and the reactive first end of the new reporter moiety, extending detection moiety 610 by another reporter moiety 106.

In some embodiments, probe 600 can be formed as a branched or tree-shaped structure in which detection moiety 610 includes multiple reporter moieties 106. For example, capture moiety 102 can be formed by a first nucleic acid having an oligonucleotide sequence, a portion of which selectively binds an analyte (such as a RNA) in a sample. Another portion of the oligonucleotide sequence of the first nucleic acid functions as part of linker 104. Probe 600 can also include a branched detection moiety 610. A portion of the branched detection moiety 610 includes an oligonucleotide sequence that hybridizes to a portion of the first nucleic acid, functioning as part of linker 104. Another portion of the branched detection moiety 610 consists of a branched oligonucleotide sequence that includes multiple reporter moieties 106, each of which includes multiple label regions to which optical labels can selectively hybridize.

A wide variety of different branched probes can be used. In certain embodiments, for example, probe 600 includes three different types of nucleic acids. The first nucleic acid is as described above, and includes an oligonucleotide sequence of which a first portion functions as capture moiety 102 and a second portion functions as a part of linker 104. The second nucleic acid is an intermediate nucleic acid. A first portion of the intermediate nucleic acid hybridizes to a portion of the first nucleic acid and functions as part of linker 104. A second portion of the intermediate nucleic acid includes multiple copies of a common oligonucleotide sequence, which function as branching hybridization locations. The probe also includes one or more third nucleic acids, each of which includes a first portion having an oligonucleotide sequence that is complementary to the common oligonucleotide sequence of the second nucleic acid, such that each third nucleic acid hybridizes to one of the common oligonucleotide sequences of the second nucleic acid. Each of the third nucleic acids also includes one or more reporter moieties, each with multiple label regions as described previously. The entire construct consisting of the first nucleic acid, the second nucleic acid, and one or more third nucleic acids, forms probe 600.

Examples of branched probes that can be used include, but are not limited to, those described in U.S. Pat. No. 9,783,841, and U.S. Patent Application Publication No. US 2015/0105298, the entire contents of each of which are incorporated herein by reference.

Because detection moiety 610 contains multiple reporter moieties 106, the optical signal that is generated when optical labels hybridize to the label regions of the multiple reporter moieties is generally more intense than optical signals that arise from single optical labels. The higher intensity optical signals allow low concentration analytes to be detected, and even single analyte molecules can be detected in this manner. Optical label hybridization, optical signal measurement, and label dehybridization and/or deactivation can generally be performed as described above.

### Compositions and Kits

The probes, reporter moieties, and optical labels described herein can be included in a variety of compositions. In some embodiments, the compositions can be used for analysis of a sample as described above in connection with FIGS. 3 and 4A-4D. Alternatively, or in addition, the compositions can be used for analysis of a sample according to different sets of steps and/or procedures.

In some embodiments, a composition—to which a sample can optionally be exposed—can include a plurality of probes. Each of the probes can include an optional capture moiety 102, an optional linker 104, and a detection moiety 610. The detection moiety 610 can include one or more reporter moieties 106. Reporter moieties 106 can have any of the label regions, and any number of label regions, described herein. The probes can also optionally include one or more verification regions 510, and any of the other features described herein.

Label regions of the reporter moieties in a composition can be selected according to the constraints described above to yield different types of probes in the composition. In particular, the composition can include groups or populations of different types of probes, where each different type of probe selectively binds to a different type of analyte in the sample. Probes of the same type each include the same reporter moieties, or optionally, reporter moieties that are indistinguishable variants of one another (i.e., reporter moieties in which the ordering of the label regions varies, but the label regions correspond to the same oligonucleotide sequences). Probes of each type have reporter moieties that differ from probes of other types of reporter moieties within the composition, without regard to the relative ordering of the label regions in each type of probe.

As discussed above, within a composition, reporter moieties can optionally be selected such that the label regions of reporter moieties of probes of a first type differ from the label regions of reporter moieties of probes of each of the other types in the composition by at least two label regions, irrespective of the relative positions of the label regions in the reporter moieties. Selecting the label regions in such a manner ensures that single add, drop, and replacement errors during hybridization and detection can be identified and corrected.

Further, reporter moieties can optionally be selected such that in a particular reporter moiety, none of the label regions is repeated. That is, for a reporter moiety with Q label regions, each of the Q label regions is distinct relative to the others in the reporter moiety. This constraint ensures that all Q label regions can be used for identification of the reporter moiety.

Compositions can generally include populations or groups of different types of probes that target any number of different types of analytes in a sample. In some embodiments, for example, compositions can include populations or groups of 2 or more (e.g., 3 or more, 4 or more, 5 or more, 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 10000 or more, or even more) different types of probes.

Each population or group of probes within a composition can generally include any number of probes of a particular type, and that selectively bind to a particular type of analyte in the sample. For example, the number of probes of a particular type can be 1 or more (e.g., 2 or more, 3 or more, 5 or more, 10 or more, 50 or more, 100 or more, 200 or more, 300 or more, 500 or more, 1000 or more, 2000 or more, 3000 or more, 5000 or more, 7000 or more, 10000 or more, 30000 or more, 50000 or more, or even more).

In some embodiments, compositions can include any of the reporter moieties described herein. The reporter moieties can be present as part of larger molecules, i.e., as part of probes or detection moieties, or alternatively, can be uncoupled to other moieties and entities within the composition. The compositions can also include reporter moieties that are present as part of detection moieties, with the detection moieties either being present as part of probes, or alternatively, uncoupled within the composition.

Label regions of the reporter moieties in such circumstances can be selected according to the constraints described above to yield different types of reporter moieties in the composition, and optionally, different types of detection moieties in the composition, and different types of probes in the composition. In particular, the composition can include groups or populations of different types of reporter moieties, where reporter moieties of the same type are the same or differ from one another merely in the relative ordering of their label regions, and reporter moieties of each type differ from reporter moieties of different types within the composition.

Within a composition, reporter moieties can optionally be selected such that the label regions of reporter moieties of a first type (e.g., reporter moieties that are part of probes of a first type) differ from the label regions of other types of reporter moieties (e.g., reporter moieties that are part of other types of probes) in the composition by at least two label regions, irrespective of the relative order of the label regions in the reporter moieties. Selecting the label regions in such a manner ensures that single add, drop, and replacement errors during hybridization and detection can be identified and corrected.

Further, reporter moieties can optionally be selected such that in a particular reporter moiety, none of the label regions is repeated. That is, for a reporter moiety with Q label regions, each of the Q label regions is distinct relative to the others in the reporter moiety. This constraint ensures that all Q label regions can be used for identification of the reporter moiety.

Compositions can generally include populations or groups of different types of reporter moieties. In some embodiments, for example, compositions can include populations or groups of 2 or more (e.g., 3 or more, 4 or more, 5 or more, 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 10000 or more, or even more) different types of reporter moieties.

Each population or group of a different type of reporter moiety within a composition can generally include any number of reporter moieties. For example, the number of reporter moieties of a particular type can be 1 or more (e.g., 2 or more, 3 or more, 5 or more, 10 or more, 50 or more, 100 or more, 200 or more, 300 or more, 500 or more, 1000 or more, 2000 or more, 3000 or more, 5000 or more, 7000 or more, 10000 or more, 30000 or more, 50000 or more, or even more).

In general, compositions can include populations or groups of different types of detection moieties, each of which includes one or more reporter moieties of the same (or indistinguishable) type. In some embodiments, for example, compositions can include populations or groups of 2 or more (e.g., 3 or more, 4 or more, 5 or more, 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 10000 or more, or even more) different types of detection moieties.

Each population or group of a different type of detection moiety within a composition can generally include any number of detection moieties. For example, the number of detection moieties of a particular type can be 1 or more (e.g., 2 or more, 3 or more, 5 or more, 10 or more, 50 or more, 100 or more, 200 or more, 300 or more, 500 or more, 1000 or more, 2000 or more, 3000 or more, 5000 or more, 7000 or more, 10000 or more, 30000 or more, 50000 or more, or even more).

In some embodiments, compositions can include one or more additional components. For example, in some embodiments, compositions can include one or more buffer solutions. Examples of suitable buffer solutions include, but are not limited to, saline-sodium citrate (SSC), phosphate-buffered saline (PBS), and tris-ethylenediaminetetraacetic acid (tris-EDTA).

In certain embodiments, compositions include one or more optical labels. The optical labels can include any of the optical labels described herein. Typically, as described in connection with FIGS. 3 and 4A-4D, a sample is exposed to optical labels in groups or pools, where each such group includes 2 or more (e.g., 3 or more, 4 or more, 5 or more, 6 or more, 8 or more, or even more) different optical labels. Within a composition, the optical labels can optionally be divided into sub-compositions, each of which contains a group or pool of optical labels. In certain embodiments, among sub-compositions, each optical label can be present in only one sub-composition. Alternatively, in some embodiments, one or more optical labels can be present in more than one sub-composition.

The sample is exposed to a composition that includes optical labels to hybridize some or all of the optical labels to complementary label regions of reporter moieties, as described above. In certain embodiments, the sample is exposed to the entire composition of optical labels at the same time. Alternatively, in some embodiments, the sample is exposed to sub-compositions of the composition sequentially as discussed previously.

In some embodiments, optical labels are present in the same composition as the probes and/or reporter moieties discussed above. In certain embodiments, optical labels are present in a different composition to which the sample is exposed after exposure to a composition that includes the probes and/or reporter moieties.

Compositions that include optical labels can also optionally include additional components. For example, in some embodiments, compositions can include one or more buffer solutions. Examples of suitable buffer solutions include, but are not limited to, saline-sodium citrate (SSC), phosphate-buffered saline (PBS), and tris-ethylenediaminetetraacetic acid (tris-EDTA).

One or more of the compositions described above can be included as part of a kit for analyzing a sample. The kit can include a housing or packaging that encloses the contents of the kit. Compositions can be contained within containers in the kit; such containers can be formed from a wide variety of materials, including (but not limited to) plastics and glass.

Kits can optionally include a variety of other components as well. For example, in some embodiments, kits can include one or more dehybridization reagents. Examples of such reagents include, but are not limited to, sodium hydroxide, dimethyl sulfoxide (DMSO), formamide, SDS, methanol, and ethanol.

In certain embodiments, kits can include one or more buffer solutions. Examples of suitable buffer solutions include, but are not limited to, saline-sodium citrate (SSC), phosphate-buffered saline (PBS), and tris-ethylenediaminetetraacetic acid (tris-EDTA).

Kits can also optionally include instructions printed or otherwise recorded on any of a variety of different media (e.g., paper, computer readable storage media) that describe the use of certain components of the kits in assays targeting analytes of various types in samples.

### Sample Analysis Systems and Components

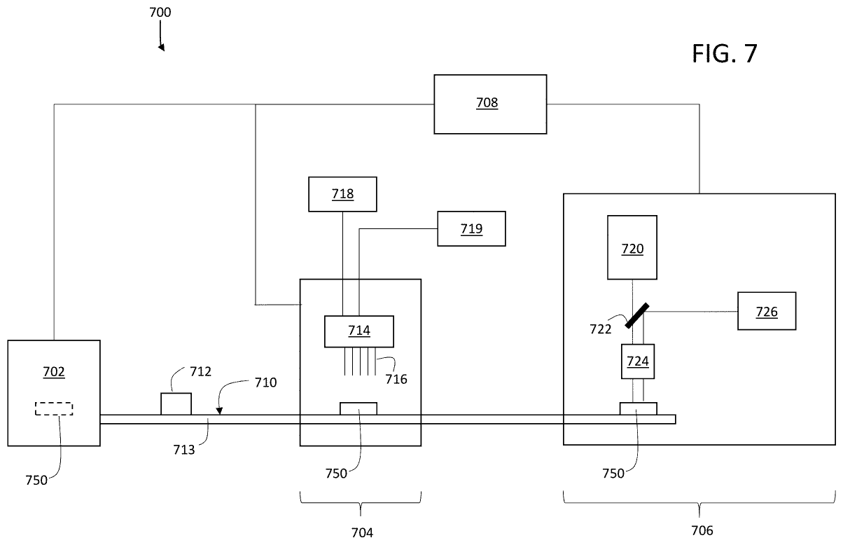

The methods described herein can be implemented in a variety of different analysis systems, including systems that perform some or all of the steps in semi-automated or fully automated fashion. One example of such a system 700 is shown schematically in FIG. 7. System 700 includes a storage unit 702, a labeling station 704, an imaging station 706, and a translation apparatus 710. Each of these components is connected to controller 708, which includes one or more electronic processors that perform control functions associated with controller 508, and can also perform any of the other analysis functions described herein.

Translation apparatus 710 includes a slide handler 712 that attaches to individual slides on which biological samples are disposed for analysis, and transport the slides between different locations in the system. An example of a slide on which a sample is disposed is indicated as slide 750 in FIG. 7. Slide handler 712 can be implemented in a number of ways. In some embodiments, for example, slide handler 712 is a grasper and includes one or more arms or fingers that exert pressure on surfaces of slide 750 to lift and transport slide 750. In certain embodiments, slide handler 712 includes a member with one or more suction ports that uses reduced pressure to lift individual slides 750. In certain embodiments, slide handler 712 includes one or more members that are inserted under slides 750 to lift the slides. In general, slide handler 712 can permit both rotational displacements of slides 750 about three orthogonal axes, and translations along three orthogonal axes.

Translation apparatus 710 can also include a track or conveyor 713 that carries individual slides 750 or containers of slides between locations in system 700. In some embodiments, track 713 is a linear track that moves back and forth along a single direction between locations. In certain embodiments, track 713 is a continuous track (e.g., circular, elliptical, or another continuous shape) that circulates among locations in the system.

During operation, controller 708 can transmit appropriate control signals to translation apparatus 710 to retrieve one or more slides 750 from storage unit 702, and to deposit one or more slides into storage unit 702, as discussed above. Further, controller 708 can transmit control signals to translation apparatus 710 to activate labeling station 704 to deliver fluids, reagents, and compositions to slide 750, and remove fluids, reagents, compositions (and components thereof) from slide 750. Labeling station 704 includes a fluidic apparatus 714 connected to one or more reservoirs 718 and to one or more pumps and/or vacuum sources 719. The fluidic apparatus 714 includes one or more fluid conduits (e.g., syringes, tubes, pipettes) are coupled to the one or more reservoirs 718, and are optionally positionable relative to slide 550, to deliver any of the reagents and/or compositions disclosed herein to the biological sample disposed on slide 550. Fluid conduits that are optionally positionable can be formed from flexible conduit materials such as any one or more of various conventional polymer materials, and coupled to robotic actuators (not shown in FIG. 7) that are connected to, and receive positioning instructions from, controller 708.

During operation of the system, controller 708 transmits signals to translation apparatus 710 to position slide 750 within labeling station 704, and transmits signals to the fluidic apparatus 714 to cause one or more of the fluid conduits to deliver fluids, reagents, and/or compositions from reservoirs 718 to the biological sample disposed on slide 550. Further controller 708 transmits signals to activate the one or more pumps and/or vacuum sources 719 to selective remove fluids, reagents, and/or compositions (and components thereof) from the biological sample. In this manner, controller 708 (transmitting instructions to labeling station 704) can implement any of the labeling, exposure, measurement, and removal/inactivation steps described herein in automated fashion.

Imaging station 706 includes a radiation source 720, an objective lens 724, a beam splitter 722, and an image detector 726. Radiation source 720 can include any one or more of a variety of different sources, including but not limited to LEDs, laser diodes, metal halide sources, incandescent sources, and fluorescent sources. Image detector 726 can include one or more different detector types, including but not limited to CCD detectors and CMOS detectors. During operation of system 700, to obtain an image of a biological sample disposed on slide 550, controller 708 activates translation apparatus 710 to position slide 750 within imaging station 706. Controller 708 then transmits control signals to the components of the imaging station, activating source 720 to deliver illumination radiation to the sample which passes through beam splitter 722 and objective lens 724 and is incident on the sample. Emitted light from the sample passes through objective lens 574, is reflected from beam splitter 722, and is incident on image detector 726, which measures an image of the emitted light.

In FIG. 7, the imaging station is configured to obtain a fluorescence or reflected-light image of the sample. However, it should be appreciated that in some embodiments, detector 726 can be positioned on an opposite side of slide 750 from source 720 to measure a transmitted-light image of the sample. In certain embodiments, imaging station 706 includes multiple detectors for measuring both transmitted- and reflected- or emitted-light (e.g., fluorescence) images of the sample. Under the control of controller 708, imaging station 706 can obtain any of the different types of images corresponding to any of the different types of dyes, stains, probes, and labeling agents described herein.

Further, it should be noted that while in the example system 700 of FIG. 7 the labeling station 704 and imaging station 706 are separate, in some embodiments the labeling and imaging stations can be combined into a single station under the control of controller 708, and which performs both sample labeling and imaging functions as described herein.

Each of the steps in sample analysis consumes a certain amount of time, and improved efficiency can be obtained by translating multiple slides 750 among multiple locations in system 700, and performing certain operations in parallel. For example, during analysis of multiple slides 750, a first slide on which a first sample is disposed can be positioned at the labeling station, and the fluidic apparatus can deliver one or more probes to the sample and incubate the sample with the delivered probes. At the same time, a second slide on which a second sample is disposed can be positioned at the imaging station to obtain one or more images of the sample. A third slide on which a third sample is disposed can be positioned at the labeling station, where controller 708 activates the one or more pumps and/or vacuum sources 719 to remove fluids, reagents, and/or compositions (and components thereof) from the sample disposed on the third slide. A fourth slide on which a fourth sample is disposed can be positioned at the labeling station, where controller 708 activates the fluidic apparatus 714 to deliver one or more fluids, reagents, and/or compositions, and incubates the fourth sample with the fluids, reagents, and/or compositions. Other slides 750 can also be processed—undergoing any of the operations discussed herein—at the same time.

As each slide reaches the end of a set of one or more steps in a preparation or analysis workflow, the slide is transported by translation apparatus 710 to a different location in the system—to a different station, or to a different location within the same station, for example. In this manner, system 700 can analyze multiple samples in parallel, ensuring that the duty cycles of the components of system 700 remain relatively high, increasing overall throughput of the system for multiple samples relative to simple linear processing of individual samples.

Additional aspects and features of system 700 are described in U.S. Patent Application Publication No. US 2020/0393343, the entire contents of which are incorporated herein by reference.

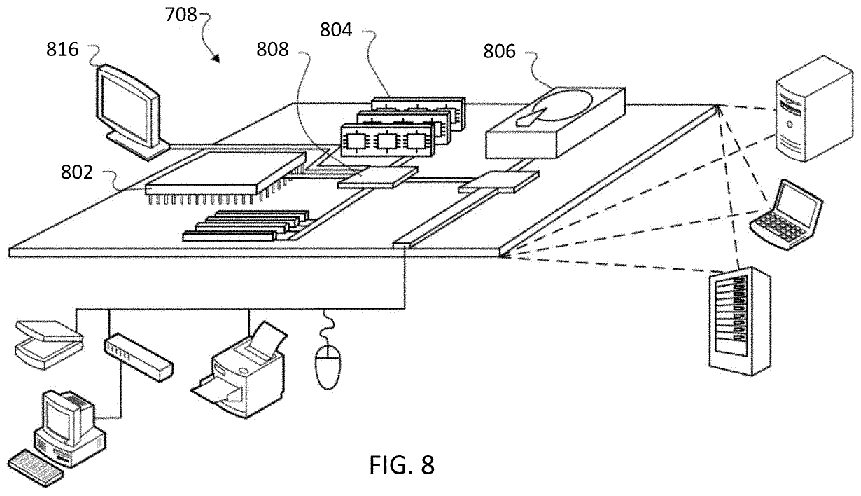

FIG. 8 shows an example of controller 708, which may be used with the systems and methods disclosed herein. Controller 708 can include one or more processors 802, memory 804, a storage device 806 and interfaces 808 for interconnection. The processor(s) 802 can process instructions for execution within the controller, including instructions stored in the memory 804 or on the storage device 806. For example, the instructions can instruct the processor 802 to perform any of the analysis and control steps disclosed herein.

The memory 804 can store executable instructions for processor 802, information about parameters of the system such as excitation and detection wavelengths, and measured image information. The storage device 806 can be a computer-readable medium, such as a floppy disk device, a hard disk device, an optical disk device, or a tape device, a flash memory or other similar solid state memory device, or an array of devices, including devices in a storage area network or other configurations. The storage device 806 can store instructions that can be executed by processor 802 as described above, and any of the other information that can be stored by memory 804.

In some embodiments, controller 708 can include a graphics processing unit to display graphical information (e.g., using a GUI or text interface) on an external input/output device, such as display 816. The graphical information can be displayed by a display device (e.g., a CRT (cathode ray tube) or LCD (liquid crystal display) monitor) for displaying any of the information, such as measured and calculated spectra and images, disclosed herein. A user can use input devices (e.g., keyboard, pointing device, touch screen, speech recognition device) to provide input to controller 708. In some embodiments, one or more such devices can be part of controller 708.

A user of system 700 can provide a variety of different types of instructions and information to controller 708 via input devices. The instructions and information can include, for example, information about any of the parameters (e.g., dyes, labels, probes, reagents, conditions) associated with any of the workflows described herein, calibration information for quantitative analysis of sample images, and instructions following manual analysis of sample images by a technician. Controller 708 can use any of these various types of information to perform the methods and functions described herein. It should also be noted that any of these types of information can be stored (e.g., in storage device 806) and recalled when needed by controller 708.

The methods disclosed herein can be implemented by controller 708 by executing instructions in one or more computer programs that are executable and/or interpretable by the controller 708. These computer programs (also known as programs, software, software applications or code) include machine instructions for a programmable processor, and can be implemented in a high-level procedural and/or object-oriented programming language, and/or in assembly/machine language. For example, computer programs can contain the instructions that can be stored in memory 804, in storage unit 806, and/or on a tangible, computer-readable medium, and executed by processor 802 as described above. As used herein, the term “computer-readable medium” refers to any computer program product, apparatus and/or device (e.g., magnetic discs, optical disks, memory, Programmable Logic Devices (PLDs), ASICs, and electronic circuitry) used to provide machine instructions and/or data to a programmable processor, including a machine-readable medium that receives machine instructions.

By executing instructions as described above (which can optionally be part of controller 708), the controller can be configured to implement any one or more of the various steps described in connection with any of the workflows herein. For example, controller 708 can selectively direct particular fluids, reagents, and/or compositions to particular regions of a biological sample (e.g., by activating fluidic apparatus 714), and selectively remove fluids, reagents, and/or compositions (and components thereof) from the sample. Controller 718 can select particular probes (with particular reporter moieties) to deliver to the biological sample, and select particular optical labels (including combinations of optical labels, according to the criteria described above) for delivery to the sample. Controller 718 can obtain measurements of optical signals from optical labels in the sample, and determine locations of the optical signals based on images received from the imaging station.

Controller 718 can determine the locations of particular analytes in the sample based on the locations of the optical signals and the types of optical labels delivered to the sample in one or more cycles of label exposure and imaging. For example, during each cycle of the workflow (see FIG. 3, for example), controller 718 can identify particular label regions present at particular locations in the sample. Based on combinations of label regions present at particular locations in the sample, controller 718 (e.g., by comparing to stored information about combinations of label regions in specific types of probes) can identify particular analytes at locations in the sample. Controller 718 can also implement error correction steps as described herein in circumstances where measured signals from optical labels cannot be unambiguously used to identify particular analytes at locations in the sample.

### Applications

The hybridization and signal detection procedures, compositions, and kits described herein can be used with a wide variety of different probes and analytes, and in a wide variety of different samples. The procedures do not depend on the manner in which analytes in the sample bind to probes, nor do they depend on the nature of the analytes. Further, other than the aspects of the label regions described herein, probes can have a wide variety of different structural compositions and functionalities. Provided they do not interfere with the hybridization of optical labels, additional structural features of the probes can be present without interfering with the procedures described herein.

Samples to which the procedures, compositions, and kits can be applied include tissue samples (e.g., tissue sections), including fresh, fresh-frozen, and formalin-fixed, paraffin-embedded (FFPE) samples. Other samples include, but are not limited to, cell and tissue lysates, body fluids (e.g., blood, urine, saliva, lymph fluid, renal fluid, and other such fluids), thick tissue (e.g., solid tumor tissue, biopsy samples), individual cells, suspensions of cells and other biological materials, and aspirated samples.

### Other Embodiments

While this disclosure describes specific implementations, these should not be construed as limitations on the scope of the disclosure, but rather as descriptions of features in certain embodiments. Features that are described in the context of separate embodiments can also generally be implemented in combination in a single embodiment. Conversely, various features that are described in the context of a single embodiment can also be implemented in multiple embodiments separately or in any suitable sub-combination. Moreover, although features may be described above as present in certain combinations and even initially claimed as such, one or more features from a claimed combination can generally be excised from the combination, and the claimed combination may be directed to a sub-combination or variation of a sub-combination.

In addition to the embodiments expressly disclosed herein, it will be understood that various modifications to the embodiments described may be made without departing from the spirit and scope of the disclosure. Accordingly, other embodiments are within the scope of the following claims.

What is claimed is:

1. A method, comprising:
(a) exposing a biological sample to a plurality of different types of probes, wherein each different type of probe comprises:
a nucleic acid capture moiety that binds to a different type of RNA analyte in the sample; and
a detection moiety comprising at least one reporter moiety, wherein the detection moiety is linked to the capture moiety, and wherein the at least one reporter moiety comprises multiple label regions, each of the label regions comprising an oligonucleotide having a sequence;
(b) exposing the biological sample to a plurality of optical labels, wherein each of the optical labels comprises an oligonucleotide having a sequence, and a species that generates an optical signal;
(c) measuring optical signals generated by optical labels of the plurality of optical labels that hybridize to complementary label regions of the reporter moieties;
(d) repeating steps (b) and (c) with different pluralities of optical labels;
(e) identifying one or more of the reporter moieties in the sample based on the measured optical signals; and
(f) determining a location of one or more of the RNA analytes in the sample based on the identified reporter moieties,
wherein the at least one reporter moiety of each type of detection moiety differs from the at least one reporter moiety of each other type of detection moiety among the different types of probes by at least two label regions.

2. The method of claim 1, further comprising, for at least one of the optical labels, removing the at least one of the optical labels from the sample after measuring an optical signal generated by the at least one of the optical labels and before repeating steps (b) and (c) with different pluralities of optical labels.

3. The method of claim 2, wherein removing the at least one of the optical labels comprises dehybridizing the at least one of the optical labels from one or more label regions of the reporter moieties.

4. The method of claim 1, wherein the at least one reporter moiety of at least one type of detection moiety comprises label regions that do not repeat within the at least one reporter moiety.

5. The method of claim 4, wherein the at least one reporter moiety of each type of detection moiety comprises label regions that do not repeat within the at least one reporter moiety.

6. The method of claim 1, wherein the at least one reporter moiety of at least one type of detection moiety comprises at least 3 label regions that do not repeat within the at least one reporter moiety.

7. The method of claim 6, wherein the at least one reporter moiety of each type of detection moiety comprises at least 3 label regions that do not repeat within the at least one reporter moiety.

8. The method of claim 1, wherein each label region comprises at least 15 nucleotides.

9. The method of claim 8, wherein each label region comprises at least 30 nucleotides.

10. The method of claim 1, wherein each label region of each of the reporter moieties comprises a same number of nucleotides.

11. The method of claim 1, wherein each of the reporter moieties comprises a same number of label regions.

12. The method of claim 1, wherein the species that generates the optical signal is a fluorescent moiety.

13. The method of claim 1, wherein the species that generates the optical signal comprises at least one fluorescent nucleotide.

14. The method of claim 1, wherein measuring optical signals generated by the optical labels comprises obtaining at least one image of the optical labels in the sample, and identifying optical signals corresponding to the optical labels in the at least one image.

15. The method of claim 1, wherein each plurality of optical labels in step (b) comprises a same number of different types of optical labels.

16. The method of claim 1, wherein each plurality of optical labels in step (b) comprises 3 or more different types of optical labels.

17. The method of claim 16, wherein each plurality of optical labels in step (b) comprises 5 or more different types of optical labels.

18. The method of claim 1, further comprising repeating step (b) until the sample has been exposed to a set of optical labels, wherein each label region of the at least one reporter moiety has a complementary optical label in the set of optical labels.

19. The method of claim 1, further comprising exposing the sample to at least one of the plurality of optical labels more than once.

20. The method of claim 1, wherein each time step (b) is performed, each member of the plurality of optical labels in step (b) comprises a species that generates a different optical signal.

21. The method of claim 20, wherein the different optical signals have different spectral distributions.

22. The method of claim 1, wherein among the plurality of optical labels:
at least two of the optical labels comprise a common species that generates the optical signal; and
the at least two of the optical labels are exposed to the sample during different repetitions of step (b).

23. The method of claim 1, wherein among the plurality of optical labels:
first and second optical labels each comprise a first species that generates the optical signals of the first and second optical labels;
third and fourth optical labels each comprise a second species that generates the optical signals of the third and fourth optical labels; and
the first and second species are different.

24. The method of claim 18, wherein the optical signals generated by the first and second species are different.

25. The method of claim 24, further comprising:
exposing the sample to the first and second optical labels during different repetitions of step (b); and
exposing the sample to the third and fourth optical labels during different repetitions of step (b).

26. A method, comprising:
exposing a biological sample to a first probe comprising a first nucleic acid capture moiety that binds to a first RNA in the sample, and a first detection moiety comprising at least one first reporter moiety, wherein the at least one first reporter moiety comprises multiple first label regions each comprising an oligonucleotide having a sequence, wherein the oligonucleotide sequences of each of the multiple first label regions in each first reporter moiety are different;
exposing the biological sample to a second probe comprising a second nucleic acid capture moiety that binds to a second RNA in the sample, and a second detection moiety comprising at least one second reporter moiety, wherein the at least one second reporter moiety comprises multiple second label regions each comprising an oligonucleotide having a sequence, wherein the oligonucleotide sequences of each of the multiple second label regions in each second reporter moiety are different;
exposing the biological sample to multiple pluralities of optical labels, wherein each plurality of optical labels comprises at least one optical label that hybridizes to at least one of the multiple first and second label regions, until optical labels from the multiple pluralities of optical labels have hybridized to each of the multiple first and second label regions;
after exposing the biological sample to each plurality of optical labels, measuring spatially resolved optical signals generated by the at least one optical label of each plurality of optical labels that hybridizes to at least one of the multiple first and second label regions; and
determining one or more locations of the first and second RNAs in the sample,
wherein at least two of the first label regions are different from each of the second label regions.

27. A composition, comprising:
multiple pluralities of probes, wherein each plurality of probes targets a different type of analyte and comprises a capture moiety that binds to the type of analyte targeted by the plurality of probes and a detection moiety linked to the capture moiety and comprising at least one reporter moiety, wherein the at least one reporter moiety comprises multiple label regions each comprising an oligonucleotide having a sequence,
wherein for each plurality of probes, the label regions of the at least one reporter moiety differ from the label regions of the at least one reporter moiety of the other pluralities of probes by at least two label regions.
